SEQUENCE LISTING (5 sequences)

SEQ ID NO: 1 - DNA, 3727 bp, Homo sapiens
aaccgccccagacacagcgctcgccgagggtcgcttggaccctgatcttacccgtgggcaccctgcgctctgcctgccgcgaagaccggctccccgacccgcagaagtcaggagagagggtgaagcggagcagcccgaggcggggcagcctcccggagcagcgccgcgcagagcccgggacaatggggccgcggcggctgctgctggtggccgcctgcttcagtctgtgcggcccgctgttgtctgcccgcacccgggcccgcaggccagaatcaaaagcaacaaatgccaccttagatccccggtcatttcttctcaggaaccccaatgataaatatgaaccattttgggaggatgaggagaaaaatgaaagtgggttaactgaatacagattagtctccatcaataaaagcagtcctcttcaaaaacaacttcctgcattcatctcagaagatgcctccggatatttgaccagctcctggctgacactctttgtcccatctgtgtacaccggagtgtttgtagtcagcctcccactaaacatcatggccatcgttgtgttcatcctgaaaatgaaggtcaagaagccggcggtggtgtacatgctgcacctggccacggcagatgtgctgtttgtgtctgtgctcccctttaagatcagctattacttttccggcagtgattggcagtttgggtctgaattgtgtcgcttcgtcactgcagcattttactgtaacatgtacgcctctatcttgctcatgacagtcataagcattgaccggtttctggctgtggtgtatcccatgcagtccctctcctggcgtactctgggaagggcttccttcacttgtctggccatctgggctttggccatcgcaggggtagtgcctctgctcctcaaggagcaaaccatccaggtgcccgggctcaacatcactacctgtcatgatgtgctcaatgaaaccctgctcgaaggctactatgcctactacttctcagccttctctgctgtcttcttttttgtgccgctgatcatttccacggtctgttatgtgtctatcattcgatgtcttagctcttccgcagttgccaaccgcagcaagaagtcccgggctttgttcctgtcagctgctgttttctgcatcttcatcatttgcttcggacccacaaacgtcctcctgattgcgcattactcattcctttctcacacttccaccacagaggctgcctactttgcctacctcctctgtgtctgtgtcagcagcataagctgctgcatcgaccccctaatttactattacgcttcctctgagtgccagaggtacgtctacagtatcttatgctgcaaagaaagttccgatcccagcagttataacagcagtgggcagttgatggcaagtaaaatggatacctgctctagtaacctgaataacagcatatacaaaaagctgttaacttaggaaaagggactgctgggaggttaaaaagaaaagtttataaaagtgaataacctgaggattctattagtccccacccaaactttattgattcacctcctaaaacaacagatgtacgacttgcatacctgctttttatgggagctgtcaagcatgtatttttgtcaattaccagaaagataacaggacgagatgacggtgttattccaagggaatattgccaatgctacagtaataaatgaatgtcacttctggatatagctaggtgacatatacatacttacatgtgtgtatatgtagatgtatgcacacacatatattatttgcagtgcagtatagaataggcactttaaaacactctttccccgcaccccagcaattatgaaaataatctctgattccctgatttaatatgcaaagtctaggttggtagagtttagccctgaacatttcatggtgttcatcaacagtgagagactccatagtttgggcttgtaccacttttgcaaataagtgtattttgaaattgtttgacggcaaggtttaagttattaagaggtaagacttagtactatctgtgcgtagaagttctagtgttttcaattttaaacatatccaagtttgaattcctaaaattatggaaacagatgaaaagcctctgttttgatatgggtagtattttttacattttacacactgtacacataagccaaaactgagcataagtcctctagtgaatgtaggctggctttcagagtaggctattcctgagagctgcatgtgtccgcccccgatggaggactccaggcagcagacacatgccagggccatgtcagacacagattggccagaaaccttcctgctgagcctcacagcagtgagactggggccactacatttgctccatcctcctgggattggctgtgaactgatcatgtttatgagaaactggcaaagcagaatgtgatatcctaggaggtaatgaccatgaaagacttctctacccatcttaaaaacaacgaaagaaggcatggacttctggatgcccatccactgggtgtaaacacatctagtagttgttctgaaatgtcagttctgatatggaagcacccattatgcgctgtggccactccaataggtgctgagtgtacagagtggaataagacagagacctgccctcaagagcaaagtagatcatgcatagagtgtgatgtatgtgtaataaatatgtttcacacaaacaaggcctgtcagctaaagaagtttgaacatttgggttactatttcttgtggttataacttaatgaaaacaatgcagtacaggacatatattttttaaaataagtctgatttaattgggcactatttatttacaaatgttttgctcaatagattgctcaaatcaggttttcttttaagaatcaatcatgtcagtctgcttagaaataacagaagaaaatagaattgacattgaaatctaggaaaattattctataatttccatttacttaagacttaatgagactttaaaagcattttttaacctcctaagtatcaagtatagaaaatcttcatggaattcacaaagtaatttggaaattaggttgaaacatatctcttatcttacgaaaaaatggtagcattttaaacaaaatagaaagttgcaaggcaaatgtttatttaaaagagcaggccaggcgcggtggctcacgcctgtaatcccagcactttgggaggctgaggcgggtggatcacgaggtcaggagatcgagaccatcctggctaacacggtgaaacccgtctctactaaaaatgcaaaaaaaattagccgggcgtggtggcaggcacctgtagtcccagctactcgggaggctgaggcaggagactggcgtgaacccaggaggcggaccttgtagtgagccgagatcgcgccactgtgctccagcctgggcaacagagcaagactccatctcaaaaaataaaaataaataaaaaataaaaaaataaaagagcaaactatttccaaataccatagaataacttacataaaagtaatataactgtattgtaagtagaagctagcactggttttattaatttagtgactattcattttatctaaatcagtgaagatttactgtcattgtttattagtctgtatatattaaaatatgatatcattaatgtacttacaaaatagtatgtcactgtttttatgttcattcttaaaaacataacctgtattaataaatgtgaacatttgcttggta

SEQ ID NO: 2 - DNA, 1275 bp, Homo sapiens
tcggtgcgtccagtggagctctgagtttcgaatcggtggcggcggattccccgcgcgcccggcgtcggggcttccaggaggatgcggagccccagcgcggcgtggctgctgggggccgccatcctgctagcagcctctctctcctgcagtggcaccatccaaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattga

SEQ ID NO: 3 - protein, 5 aa, Artificial
  synthetic polypeptide
TFLLR

SEQ ID NO: 4 - protein, 6 aa, Artificial
  synthetic polypeptide
SLIGKV

SEQ ID NO: 5 - protein, 6 aa, Artificial
  synthetic polypeptide
  MOD_RES (1)..(1): Leu at residue 1 is a 2-furoyl-Leu
  MOD_RES (6)..(6): Orn
LIGALX

(19) United States
(12) Patent Application Publication
(10) Pub. No.: US 2023/0061116 A1
(43) Pub. Date: Mar. 2, 2023

(54) TARGETING PAR1 AND PAR2 TO REGULATE LIPID AND CHOLESTEROL ABUNDANCE

(71) Applicant: Mayo Foundation for Medical Education and Research, Rochester, MN (US)

(72) Inventors: Isobel A. Scarisbrick, Rochester, MN (US); Erin M. Triplet, Rochester, MN (US)

(21) Appl. No.: 17/792,591

(22) PCT Filed: Jan. 13, 2021
(86) PCT No.: PCT/US2021/013251

§ 371 Date: Jul. 13, 2022

Related U.S. Application Data
(60) Provisional application No. 62/960,881, filed on Jan. 14, 2020.

(51) Int. Cl. A61K 38/08; A61K 31/443; A61K 31/438; A61K 31/136; A61P 3/06

(52) U.S. Cl. CPC A61K 38/08; A61K 31/443; A61K 31/438; A61K 31/136; A61P 3/06

(57) ABSTRACT

Materials and methods for regulating lipid abundance by modulating Protease Activated Receptor 1 (PAR1) and Protease Activated Receptor 2 (PAR2) levels arc provided herein. In a first aspect, this document features a method that includes (a) identifying a mammal as having a condition characterized at least by impaired lipid production, impaired cholesterol production, or both impaired lipid production and impaired cholesterol production; and (b) administering to the mammal an inhibitor of PAR1 and/or PAR2 in an amount effective to increase lipid production and/or cholesterol production in the mammal.

### CROSS-REFERENCE TO RELATED APPLICATIONS

This application claims benefit of priority from U.S. Provisional Application Ser. No. 62/960,881, filed Jan. 14, 2020. The disclosure of the prior application is considered part of (and is incorporated by reference in) the disclosure of this application.

### TECHNICAL FIELD

This document relates to materials and methods for regulating lipid abundance by modulating Protease Activated Receptor 1 (PAR1) and Protease Activated Receptor 2 (PAR2) levels.

### BACKGROUND

Lipids and cholesterol play key structural and signaling roles in the brain, spinal cord, and all other tissues and organs of the body. The central nervous system is especially cholesterol rich (containing ˜20% of the body's cholesterol) and is particularly vulnerable to disorders of lipid and cholesterol synthesis. Identification of factors regulating lipid and cholesterol metabolism therefore can be seen as essential to understanding human physiology/pathophysiology, and to the identification of new therapies targeting cholesterol and lipid production that will ultimately have wide clinical utility.

### SUMMARY

This document is based, at least in part, on the discovery that blocking the thrombin receptor, also known as PAR1, can increase biosynthesis of lipids and cholesterol. This document also is based, at least in part, on the discovery that blocking PAR2 can increase lipid and cholesterol biosynthesis. Thus, blocking PAR1 and/or PAR2 provides a therapeutic strategy for increasing lipid production, while activating PAR1 and/or PAR2 provides a strategy for inhibiting lipid production and cholesterol biosynthesis.

In a first aspect, this document features a method that includes (a) identifying a mammal as having a condition characterized at least by impaired lipid production, impaired cholesterol production, or both impaired lipid production and impaired cholesterol production; and (b) administering to the mammal an inhibitor of PAR1 and/or PAR2 in an amount effective to increase lipid production and/or cholesterol production in the mammal. The mammal can be a human. The condition can be selected from the group consisting of Alexander disease, metachromatic leukodystrophy, Krabbe disease, adrenoleukodystrophy, Canavan disease, Pelizaeus-Merzbacher disease, cerebrotenineous xanthomatosis, hypomyelinating leukodystrophy type 7, Refsum disease, and congenital lipidoses. The inhibitor of PAR1 and/or PAR2 can be a small molecule (e.g., Vorapaxar, GB88, or a parmodulin).

In another aspect, this document features a method that includes (a) identifying a mammal as having a condition characterized at least by excess lipid production, excess cholesterol production, or both excess lipid production and excess cholesterol production; and (b) administering to the mammal an activator of PAR1 and/or PAR2 in an amount effective to reduce lipid production and/or cholesterol production in the mammal. The mammal can be a human. The condition can be selected from the group consisting of Smith-Lemli-Opitz Syndrome, Gaucher, Niemann-Pick, Tay-Sachs, fatty liver disease, familial hypercholesteroletnia, adrenoleukodystrophy, Fabry, Farber, Hunter, Hurler, Krabbe, Tangier, acquired dyslipidemia, and non-alcoholic fatty liver disease (NAFLD). The activator of PAR1 and/or PAR2 can be a peptide ligand (e.g., Thr-Phe-Leu-Leu-Arg (SEQ ID NO:3), Ser-Leu-Ile-Gly-Lys-Val (SEQ ID NO:4), or 2-Furoyl-Leu-Ile-Gly-Arg-Leu-Orn (SEQ ID NO:5)).

In another aspect, this document feature a method that includes administering an inhibitor of PAR1 and/or PAR2 to a mammal identified as having a condition characterized at least by impaired lipid production, impaired cholesterol production, or both impaired lipid production and impaired cholesterol production, where the inhibitor is administered in an amount effective to increase lipid and/or cholesterol production in the mammal. The mammal can be a human. The condition can be selected from the group consisting of Alexander disease, metachromatic leukodystrophy, Krabbe disease, adrenoleukodystrophy, Canavan disease, Pelizaeus-Merzbacher disease, cerebrotenineous xanthomatosis, hypomyelinating leukodystrophy type 7, Refsum disease, and congenital lipidoses. The inhibitor of PAR1 and/or PAR2 can be a small molecule (e.g., Vorapaxar, GB88, or a parmoldulin).

In still another aspect, this document features a method that includes administering an activator of PAR1 and/or PAR2 to a mammal identified as having a condition characterized at least by excess lipid production, excess cholesterol production, or both excess lipid production and excess cholesterol production, where the activator is administered in an amount effective to reduce lipid production and/or cholesterol production in the mammal. The mammal can be a human. The condition can be selected from the group consisting of Smith-Lemli-Opitz Syndrome, Gaucher, Niemann-Pick, Tay-Sachs, fatty liver disease, familial hypercholesterolemia, adrenoleukodystrophy, Fabry, Farber, Hunter, Hurler, Krabbe, Tangier, acquired dyslipidemia, and NAFLD. The activator of PAR1 and/or PAR2 can be a peptide ligand (e.g., Thr-Phe-Leu-Leu-Arg (SEQ ID NO:3), Ser-Leu-Ile-Gly-Lys-Val (SEQ ID NO:4), or 2-Furoyl-Leu-lle-Gly-Arg-Leu-Orn (SEQ ID NO:5)).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B provide results from whole genome RNA sequencing of the spinal cord of adult PAR1+/+ and PAR1−/− mice. These studies revealed that PAR1 loss-of-function increased expression of genes essential for cholesterol and lipid production and neural cell differentiation (FIG. 1A). PANTHER Gene Ontology (GO) pointed to cholesterol biosynthesis as one of the top pathways affected, along with axon ensheathment and myelination (FIG. 1B; see, also, FIG. 2 for Liquid Chromatography-Mass Spectrometry (LC-MS) quantification of lipids). Hmgcsl codes for an enzyme involved in the production of HMGCoA, a rate-limiting step in cholesterol biosynthesis. DHCR7 codes for an enzyme involved in the conversion of 7-dehydrocholesterol to cholesterol with mutations disrupting cholesterol synthesis manifesting in Smith-Lemli-Opitz Syndrome (Saher et al., Biochim Biophys Acta. 2015, 1851(8):1083-1094; and Berghoff et al., Nature Commun 2017, 8:14241). Ugt8 (galactosyltransferase) and Fa2h (fatty acid 2-hydroxylase) are essential components of lipid synthesis pathways critical for membrane formation (FDR P<0.05, n=4).

FIG. 2 is a series of graphs plotting levels of cholesterol, sphingomyelin, and sphingolipid in the spinal cord of PAR1+/+ and PAR1−/− mice at postnatal days 21 (P21) and 60 (P60), as determined by LC-MS. These studies showed increased cholesterol and sphingomyelin in the spinal cord of PAR1−/− mice at P21, and increased sphingolipid at P60. Brain and liver also were assessed using tissues collected from the same mice.

FIG. 3 is a graph plotting HMGCS1 expression in cultures of primary cortical neurons treated with the PAR1 small molecule inhibitor Vorapaxar (100 nM) or vehicle control for 72 hours. These studies demonstrated that Vorapaxar promoted increases in HMGCS1 expression, compared to vehicle-treated controls (*P<0.05, Student's t-test).

FIGS. 4A-4D show differential gene expression in whole genome RNA sequencing of the spinal cord of adult PAR1+/+ (black bars), PAR1−/− (light gray bars) or PAR2−/− (dark gray bars) mice, demonstrating that PAR gene knockout (KO) increases expression of genes critical for myelination, cholesterol and lipid biosynthesis (n=4 per genotype, FDR P<0.05). Genes that were upregulated in PAR1−/− mice included those encoding the major myelin proteins PLP1 and MBP (FIG. 4A), Hmgcs1(FIG. 4C), which codes for an enzyme involved in production of HMG-CoA (a rate-limiting step in cholesterol biosynthesis), and DHCR7 (FIG. 4C), which encodes an enzyme involved in the conversion of 7-dehydrocholesterol to cholesterol, as well as Ugt8 (galactosyltransferase) and Fa2h (fatty acid 2-hydroxylase) (FIG. 4A), which are essential components of lipid synthesis pathways critical for membrane formation. PANTHER GO Pathways highlighted key myelination events, including axon ensheathment, lipid biosynthesis, and oligodendrocyte differentiation (uninjured (UI) animals shown, FIG. 4B). Ingenuity Pathways analysis highlighted signaling to intermediates in the ERK and AKT pathways (FIG. 4D) that was confirmed by Western analysis.

FIG. 5 is a series of graphs plotting levels of cholesterol, sphingomyelin, and sphingolipid in the spinal cord of PAR1 and PAR2 knockout mice, showing that PAR1 and PAR2 knockout mice exhibit higher levels of cholesterol and/or lipid synthesis. In particular, cholesterol and sphingomyelin were increased in the spinal cord of PAR−/− mice at P21, and sphingolipid was increased at P60. The spinal cord of PAR2−/− mice showed increases in sphingomyelin at P21 and P60 relative to PAR+/+ (*P<0.05, **P<0.01, ***P<0.001, NK). All lipids were quantified by LC-MS.

FIGS. 6A-6G show that PART knockout mice exhibited improved myelin regeneration in a lysolecithin model of focal demyelination. FIG. 6A is a table providing the mean number of remyelinated axons after focal lysolecithin-mediated demyelination of the ventral spinal cord white matter (FIG. 6B) in PAR+/+ and PAR1−/− mice. The number of remyelinated axons was greater in PART knockout mice at 14 or 28 days post injury (dpi) (P=0.007 and P=0.03, Student's t-test). FIG. 6C is a series of images showing representative paraphenylenediamine-stained thin sections from which the counts of remyelinated axons were made (left panel), and a graph plotting the numbers of remyelinated axons (right panel). FIGS. 6D-6G are a series of images and graphs showing that PAR1 knockout mice exhibited improvements in the number of Olig2+ oligodendrocyte lineage cells at 14 and 28 dpi (FIG. 6D; P=0.001 and P=0.04) and increases in the number of CC-1+ mature oligodendrocytes at 28 dpi (FIG. 6E). By 28 dpi, higher levels of MBP were identified in the lesion area of PAR1 knockout mice (FIG. 6F; P=0.05). No significant changes were observed in neurofilaments across time or genotype (FIG. 6G). Bar graphs at the right in FIGS. 6D-6G represent mean±S.E.M. of PAR1+/+, n=7 at 14 d and n=9 at 28 d; PAR1−/− n=8 at 14 d and n=10 at 28 d. Asterisks in FIGS. 6D-6G represent significant differences with *P<0.05; **P<0.01; ***P≤0.001, Student's t-test. Scale bars indicate 20 μm (FIG. 6C) and 50 μm (FIGS. 6D-6G).

FIG. 7 shows that PAR1 knockout improves the cholesterol synthesis pathway in spinal cord oligodendrocytes after focal demyelinating injury. Hmges1 is an essential regulatory enzyme in cholesterol production. The graphs plot the percentage of Hmgsc1+ Olig2+ cells, showing that Hmgsc1 was expressed at higher levels by Olig2+ oligodendrocytes in PAR1 knockout mice at 14 and 28 days post lysolecithin injury (dpi) compared to wild type controls. Bar graphs represent mean±S.E.M. of PAR1+/+, n=7 at 14 d and n=8 at 28 d; PAR2−/− n=7 at 14 d and n=6 at 28 d. (*P<0.05 Student-Newman-Keuls (SNK)).

FIG. 8 is a pair of graphs demonstrating that PAR1 knockout mice have increased expression of SREBP1 (left panel) and SREBP2 (right panel) in astrocytes during myelin repair following lysolecithin-induced demyelination in the spinal cord. SREBP1 and SREBP2 are master transcriptional regulators of cholesterol and lipid biosynthesis. Spinal cords were stained for GFAP (astrocyte marker) and SREBP1 and 2 at 14 or 28 days following lysolecithin-mediated focal demyelination. SREBP positivity was defined by measuring the percent of GFAP+ area colocalized with SREBP within the demyelinated lesion. SREBP2 expression in astrocytes was significantly increased in PAR1 knockout compared to wild-type controls at 14 days; both SREBPs trended toward higher astrocytic expression at all time points in the context of PAR1 knockout (*P<0.05, SNK, n=6 B6 14d, 5 PAR1 14d, 5 B6 28d, 8 PAR1 28d).

FIGS. 9A-9F show that PAR1 knockout improves remyelination after cuprizone (CPZ) withdrawal. FIG. 9A is a schematic depicting the phases of demyelination and remyelination during and after CPZ feeding, with arrows indicating time points for rotarod assessment (FIG. 913) and immunohistochemistry (IHC) for markers of myelin injury and repair (FIGS. 9C-9F). The impact of PAR1 knockout on remyelination was assessed by feeding mice CPZ-laden chow for 6 weeks, followed by a period of “induced remyelination” upon CPZ withdrawal and feeding regular chow for an additional 3 (6+3) or 6 (6+6) week period. FIG. 9B is a graph plotting motor function as assessed by angular speed at fall on an accelerating rotarod test, expressed as a percent of maximum at baseline for each genotype. These studies demonstrated less CPZ-related deficit at 3 (P=0.001) and 6 weeks (P=0.006) of CPZ-feeding and after an additional 3 weeks on normal chow (6+3) in PARA knockout mice (P=0.003). FIGS. 9C-9F provide representative images through the corpus callosum of mice after 3 or 6 weeks of remyelination (6+3 or 6+6). Corresponding quantification of markers of remyelination showed that PAR1−/− mice exhibited greater increases in the number of oligodendrocyte lineage cells (Olig2+) (FIG. 9C) after 3 weeks on regular chow and in the number of mature oligodendrocytes (CC-1+) (FIG. 9D) at 3 and 6 weeks, compared to wild type (P≤0.05). The area stained for MBP or neurofilament (NF) after CPZ, treatment was not altered by PAR1 knockout (FIG. 9E). However, at the 6+6 week time point, PAR1−/− mice consuming regular chow for the full period of the experiment showed higher counts of CC-1+ and higher levels of MBP and NF immunoreactivity in the corpus callosum (P≤0.04) relative to regular chow WT counterparts (FIGS. 9D, 9E, and 9F). Statistical evaluation of motor function (FIG. 9B) was done by two-way ANOVA followed by Neuman Keuls post hoc test (PAR1−/− effect P<0.003, n=4-8 for each genotype). Each bar in the graphs of FIGS. 9C-9F represents the mean value from n=4 to 5 for each genotype across diet conditions with individual data points shown, error bars±S.E.M. Asterisks (FIGS. 9C-9F) represent significant differences with *P<0.05; **P<0.01; ***P≤0.001, Student's t-test. Scale bar=100 μm.

FIGS. 10A-10D show that PAR1 inhibition increases oligodendrocyte expression of cholesterol synthesis machinery during CNS myelin regeneration, and that enhanced remyelination after cuprizone-mediated demyelination in PAR1 knockout mice was accompanied by increases in the percentage of HMGCS1+ oligodendrocytes after acute lysolecithin- or chronic cuprizone-mediated demyelination. FIG. 10A includes a series of representative images of the ventrolateral spinal cord at 14 or 28 days following lysolecithin injection, demonstrating co-labelling of Olig2 for oligodendrocyte lineage cells and the cholesterol synthesis enzyme HMGCS1. The area of demyelination is outlined with a white dashed line. PAR1−/− mice showed significantly more HMGCS1-expressing oligodendrocytes at 28 (p=0.001) days of remyelination. The percentage of HMGSC1+ cells is plotted in the histogram shown in FIG. 10B, with values representing the mean±S.E.M. PAR1+1+, n=7 at 14 days and n=9 at 28 days; PAR1−/−, n=8 at 14 days and n=10 at 28 days, male mice. FIG. 10C includes representative images taken in the corpus callosum after 6 weeks of cuprizone feeding to induce demyelination, followed by 3 weeks of feeding regular chow to elicit remyelination. Cells were co-labeled with Olig2 and HMGCS1. Oligodendrocyte expression of HMGCS1 was increased in both wild type (p<0.001) and PAR1−/− (p<0.001) after 6 weeks of cuprizone feeding plus 3 weeks on a regular diet to induce repair (6+3), with PAR1−/− showing greater increases in the number of HMGCS1+Olig2+ cells compared to wild type (p<0.001). The data plotted in the graph shown in FIG. 10D represent mean values for the percentage of HMGSC1+ cells from n=4 to 5 male mice for each genotype across diet conditions (error bars, ±S.E.M.). *p<0.05, Two-Way ANOVA. Scale bar=50 μM.

FIG. 11 contains a series of images showing that Hmgcs1 is expressed at higher levels by NeuN⁺ neurons in the cingulate cortex in PAR1 knockout mice prior to 6 weeks of CPZ mediated injury (Ctrl) and after 3 weeks on a regular diet to elicit repair (6+3). These results suggested that blocking PAR1 increases cholesterol synthesis in neurons in the intact CNS and in response to CPZ mediated injury. Results are plotted in the graph in the right panel (*P<0.05 SNK).

FIG. 12 contains a series of images showing that inhibition of PAR1 signaling accelerates neurite outgrowth in primary mouse cortical neurons. Murine neurons were cultured for 24 hours before addition of the PAR1 inhibitor Vorapaxar, subtherapeutic growth factor BDNF, or both. Inhibition of PAR1 potentiated the effect of BDNF, significantly increasing production of neurites at both 24 and 72 hours of treatment. Neurite outgrowth was quantified by staining for TUJI (a cytoskeletal protein present in both axons and dendrites) and measuring TUJI+ area (the average of 5 randomly selected fields per well, 6 wells per treatment). Production of lipids and cholesterol, major constituents of neuronal membranes, was required to grow both axons and dendrites. Results are plotted in the graphs in the right panel (*P<0,05 SNK, n=6 all groups, both timepoints).

FIG. 13 contains images showing that inhibition of PAR1 signaling accelerates synaptogenesis in primary mouse cortical neurons. Murine neurons were cultured in vitro for 24 hours before addition of the PAR1 inhibitor Vorapaxar, subtherapeutic growth factor BDNF, Or both. Inhibition of PAR1 potentiated the effect of BDNF, significantly increasing production of synaptic densities at both 24 and 72 hours of treatment. Synapse density was quantified by staining for Homer, a scaffold protein concentrated at the post-synaptic densities by counting puncta in ImageJ. Synapse counts were normalized by total neurite area (TUJ1+ area) to determine the density of synapses on neurites (average of 5 randomly selected fields per well, 6 wells per treatment). Results are plotted in the graphs in the right panel *P<0.05 SNK, n=6 all groups, both timepoints).

FIG. 14 contains a series of images showing that inhibition of PAR1 signaling accelerates growth of new neurites following transection injury in vitro. Murine neurons were cultured in vitro to confluence, and then a large wound was created by mechanically scratching the center of each field. The PAR1 inhibitor Vorapaxar, subtherapeutic growth factor BDNF, or both were added following injury. Inhibition of PAR1 potentiated the effect of BDNF, significantly increasing growth of new neurites through the injury zone, measured by TUJ1+ area in the scratch (the average of 3 randomly selected fields per well, 6 wells per treatment). Results are plotted in the graph in the right panel (*P<0.05 SNK, n=6 all groups).

FIG. 15 is a graph plotting expression of expression of HMGCS1, and demonstrating that inhibition of PAR1 by Vorapaxar increases expression of cholesterol synthesis enzymes in primary murine cortical neurons. Expression of HMGCS1, an enzyme responsible for production of HMGCoA for the rate-limiting step in cholesterol biosynthesis, was quantified by real-time quantitative PCR. Inhibition of PAR1 (with or without co-treatment with BDNF) increased the expression of HMGCS1 in cortical neurons. *P<0.05 SNK; n=S for BDNF, n=6 for all other groups.

FIG. 16 contains a series of images showing that inhibition of cholesterol production with statins (reversible inhibitors of HMG-CoA Reductase) diminishes neurite outgrowth of primary mouse cortical neurons, demonstrating the essential role of cholesterol production in neuron growth and development. Inhibition of PAR1 (by Vorapaxar) or growth factor addition (by BDNF) alone did not result in recovery of neurite production and still produced a statistically significant decrease in neurite production compared to controls. However, co-treatment with both subtherapeutic BDNF with inhibition of PAR1 partially rescued neurite production from statin treatment, and cotreated cultures were no longer statistically different from untreated controls. Neurite outgrowth was quantified by staining for TUJ1 (a cytoskeletal protein present in both axons and dendrites) and measuring TUJ1+ area (the average of 5 randomly selected fields per well, 6 wells per treatment). *P<0.05 SNK, n=6 all groups.

FIG. 17 is a graph plotting neurite area for primary mouse cortical neurons following mechanical injury, showing that inhibition of cholesterol production with statins diminished neurite outgrowth, and demonstrating the essential role of cholesterol production in neuron repair as well as development. Co-treatment with both subtherapeutic BDNF and inhibition of PAR1 rescued neurite repair from statin treatment and additionally increased repair even beyond untreated control cultures. Neurite outgrowth was quantified by staining for TUJ1 in the scratch zone and measuring TUJ1+ area (the average of 3 randomly selected fields per well, 6 wells per treatment). *P<0.05 SNK, n=3 for all groups.

FIGS. 18A-18D show that PAR1 knockout promotes increased expression of cholesterol synthesis intermediates by neurons in the injured spinal cord. FIG. 18A contains a series of immunofluorescent images showing co-labeling for NeuN (neuron marker) and HMGCS1 in spinal segments above the injury epicenter 30 days after 0.25 mm lateral compression (LC) and FEJOTA clip contusion-compression SCI in wild type (PAR1+/+) and PAR1−/− mice. The merged images show higher power views of boxed areas to demonstrate increases in neuronal HMGCS1 expression in PAR1−/− compared to controls after SCI (arrow heads indicate HMGCS1+NeuN+ventral horn neurons). Scale bar=100 μM. FIG. 18B includes histograms plotting the number of HMGCS1+ neurons in spinal segments above and below the injury epicenter in the PAR1+/+ and PAR1−/− mice. There were significant elevations in the number of HMGCS1+ neurons 30 dpi (p=<0.001 Above for LC, p=0.002 Below for LC, p=0.04 Above for FEJOTA). Few HMGCS1+ neurons were present in the intact (uninjured) spinal cord. FIG. 18C includes histograms plotting the total number of neurons in the spinal cord, which did not significantly differ by genotype in either injury. FIG. 18D includes images showing expression of HMGCS1 by ventral horn motoneurons in intact human spinal cord and at subacute and chronic time points after traumatic SCI. Scale bar=50 μM. For the experiments illustrated in FIGS. 18A-18D, differences between genotypes were measured by two-tailed Student's t-test (uninjured) or Two-Way Repeated Measures ANOVA with Student-Newman-Keuls pairwise comparisons (SCI), *p<0.05. n=7 PAR1+/+ and 6 PAR1−/− female mice for LC SCI, n=8 PAR1+/+ and n=7 PAR1−/− mice for FEJOTA SCI, n=3 each genotype for uninjured immunostaining. Co-expression between uninjured and SCI (average of all segments) also was assessed by Two-Way ANOVA; differences between intact and SCI were not significant in either genotype.

FIG. 19 is a graph showing that a higher percentage of neurons in PAR1−/− uninjured spinal cord express HMGCS1 than wild type controls at baseline. Spinal cord sections (5 per animal) were imaged at levels corresponding to the SCI tissue (above, epicenter, below) and HMGCS1+ NeuN+ neurons counted in Imaga P<0.05, n=3 for both groups.

FIGS. 20A-20D show that enhanced remyelination after lysolecithin demyelination in PAR1−/− knockout mice was accompanied by increases in the number of SREBP1+ and SREPB2+ oligodendrocyte lineage cells. FIG. 20A includes a series of representative images of remyelinating lesions at 14 or 28 days after lysolecithin microinjection into the ventrolateral spinal cord white matter of wild type or PAR1 knockout mice, co-labeled with Olig2 for oligodendrocyte lineage cells and SREB1. Lesion borders are outlined by a dashed line. FIG. 20B is a graph plotting the corresponding quantification (mean±S.E.M.), showing the increases in SREBP1+ oligodendrocytes overall in PAR1−/− mice compared to wild type during remyelination (p=0.02). FIG. 20C is a series of representative images of remyelinating lesions at the same time points as in FIG. 20A, which were co-stained for Olig2 and SREBP2. Two-Way ANOVA demonstrated a significant genotype difference, with PAR1−/− showing significantly more SREBP2+ Olig2+ cells in remyefinating lesions (p=0.04). FIG. 20D is a histogram plotting the data (mean±SEM) from FIG. 20C. PAR1+1+, n=7 at 14 days and n=9 at 28 days; PAR1−/−, n=8 at 14 days and n=10 at 28 days, male mice. Significance was determined by Two-Way ANOVA, *p<0.05. Scale bar=50 μM.

FIGS. 21A-21D show that enhanced remyelination after cuprizone-mediated demyelination in PAR1−/− mice was accompanied by increases in the number of SREBP1+ and SREBP2+ oligodendrocyte lineage cells. FIG. 21A includes a series of representative images of SREBP1 and SREBP2 co-localization with Olig2 in the corpus callosum on regular chow (untreated) and after 6 weeks of CPZ feeding to induce demyelination followed by a 3 week period of regular chow consumption to induce myelin regeneration. Scale bar=50 μM. The corresponding quantification (FIG. 21B) demonstrates increases in SREBP1+ oligodendrocytes during remyelination in both wild type and PAR1−/− mice (p=0.006 and p<0.001, respectively), with the greatest increases observed in mice lacking PAR1−/− (p<0.001 compared to wild type). FIG. 21C is a series of representative images through the corpus callosum at the same time points as in FIG. 21A, co-stained for Olig2 and SREBP2, and demonstrating a similar pattern of increased SREBP2+ oligodendrocytes in response at 3 weeks of myelin regeneration. Scale bar=50 μM. The greatest increases were observed in mice lacking PAR1. PAR1 knockout mice also showed a greater number of SREBP2+ Olig2+ oligodendrocyte lineage cells in age matched controls that were not fed cuprizone (p=0.02) and during post-cuprizone remyelination (p=0.004), Data are plotted in the histogram of FIG. 21D, which show the mean value from n=4 to 5 male mice for each genotype across diet conditions (±S.E.M.). Significance was determined by Two-Way ANOVA, *p<0.05.

FIG. 22 is a graph plotting human neuronal (SH-SY5Y) cells grown in the presence of the small molecule PAR1 inhibitor, Vorapaxar (“Vora,” at 100 nM) or control for 72 hours. Cells treated with the inhibitor contained more than twice the amount of cellular cholesterol than untreated cells. Cholesterol content was quantified by the amplex red colorimetric assay.

### DETAILED DESCRIPTION

PAR1 and PAR2 are G protein coupled receptors that are specifically activated by select serine proteases. Under physiological conditions, these receptors communicate changes in the proteolytic microenvironment to cells, activating or inhibiting intracellular signaling cascades that modulate cellular physiology. Depending on the cell type, PAR1 activation engages several Ga subunits, Gβγ, G12/13, Gq/1.1. or Gi resulting in modulation of signaling through Rho.GEF, P1-PLC, MAPK, PI3-kinase or adenylate cyclase pathways. PARs therefore can serve as biosensors that translate dynamic changes in the proteolytic microenvironment into adaptive (or maladaptive) cellular responses. Studies described elsewhere have demonstrated that blocking the function of PAR1 or PAR2 can speed myelin development (Yoon et al., Glia 2015, 63(5):846-859; and Yoon et al., Glia 2017, 65(12):2070-2086) and improve myelin regeneration in acute (lysolecithin) and chronic (CPZ) models of myelin injury and regeneration (Yoon et al. 2017, supra). PAR1 and PAR2 knockout mice also show superior recovery of function after traumatic spinal cord injury (Ra.dulovic et al., Neurobial Dis 2015, 83:75-89; and Radulovic et al., Neurohiol Dis 2016, 93:226-242), including improved recovery of myelin (Yoon et al. 2017, supra) and synaptic elements.

As described in the Examples herein, quantification of lipid content in the central nervous system (CNS) of wild type and PAR1 knockout mice revealed increases in lipid availability in the knockout mice, including availability of hound cholesterol, galactosylceramides, sphingolipids, and sphingomyelins. Proteomic analysis demonstrated that blocking PAR1 increased ApoA1, the primary apolipoprotein of high-density lipoprotein (HDL). In addition, PANTHER GO analysis of RNA sequencing data obtained from the CNS of wild type compared to PAR1 knockout mice demonstrated that mice with knockout of the PAR1 gene had increases in lipid and cholesterol biosynthetic processes. Blocking PAR1 increased expression of genes involved in cholesterol biosynthesis pathways, including the Hmges1, Sqle, Mvd, Lss. Dher7, Fdft1, and Hmger genes.

Also as described herein, PANTHER GO analysis of RNA sequencing data obtained from the CNS of wild type mice and PAR2 knockout mice demonstrated that mice with PAR2 loss-of-function had increases in sterol and cholesterol biosynthetic processes. Blocking PAR2 increased the expression of genes involved in cholesterol biosynthesis, including the Hmgcs1, Sqle, Mvd, Lss, Dhcr7, Fdft1, and Hmgcr genes. Blocking PAR2 therefore can increase the availability of lipids, including the fatty acid DHA, sphingolipids, and sphingomyelin.

In some embodiments, this document provides methods and materials for targeting PAR1 and/or PAR2, which can have clinical utility in the treatment of disorders of lipid synthesis. Disorders that result from impaired production of lipids necessary for normal brain development (frequently referred to as congenital dysmyelinating disorders) include, without limitation, Alexander disease, metachromatic leukodystrophy, Krabbe disease, adrenoleukodystrophy, Canavan disease, Pelizaeus-Merzbacher disease, cerebrotenineous xanthomatosis, hypomyelinating leukodystrophy type 7, and Refsum disease. These conditions share an underlying impairment in normal lipid production, and can result in a wide range of clinical phenotypes, including seizures, progressive weakness, and lack of formation of myelin needed to insulate nerves for CNS communication. In some cases, targeting PAR1 and/or PAR2 (e.g., with one or more small molecule inhibitors) can lead to increased expression of lipid synthesis genes, allowing for compensation of disease-related defects through elevated production of related lipid species.

In some cases, a small molecule inhibitor of PAR1 and/or PAR2 can be used. Small molecule inhibitors can be orally bioavailable and can have moderate penetrance through the blood-brain barrier, providing a strong rationale for successful treatment with long-term oral administration of these inhibitors. Since disorders of lipid synthesis typically are congenital disorders, the treatment paradigm may necessitate life-long administration, with early diagnosis and therapy initiation most likely to produce positive results. Reducing the activity of PAR1 or PAR2 to increase lipid and cholesterol levels also may provide benefit for conditions in which increases in lipids and cholesterol will enhance tissue regeneration (e.g., demyelinating lesions, neurotrauma, neurodegeneration, and congenital lipidoses).

In some cases, activation of PAR1 and/or PAR2 signaling also can have clinical benefits. For example, Smith Lemli Opitz is a severe disorder caused by mutation in cholesterol synthesis enzymes. The mutation causes not only reduced production of cholesterol (which may be supplemented exogenously), but also toxic buildup of cholesterol production byproducts that accumulate over time, which manifests with multiple system abnormalities across the brain and peripheral organs. Activation of PAR1 and/or PAR2 with, for example, targeted short peptide ligands, can diminish cholesterol to production in vivo, reducing the buildup of toxins and ameliorating the disease phenotype. Disturbances in lipid and cholesterol metabolism also contribute to a number of other health conditions. For example, in Gaucher, Niemann-Pick, and Tay-Sachs, excess lipid accumulates in the brain and/or in peripheral organs, including the liver, heart, lungs and kidneys. Individual organs can also be subject to excess lipid accumulation, such as in fatty liver disease. High circulating cholesterol levels also is a major risk factor for heart disease and stroke, the leading causes of death in the United States. Increasing the expression of activity of PAR1 or PAR2 to reduce lipid and cholesterol levels may provide a new treatment target for disorders such as those listed above, as well as other clinical conditions [e.g., familial hypercholesterolemia, adrenoleukodystrophy, Fabry, Farber, Hunter, Hurler, Krabbe, Tangier, and non-alcoholic fatty liver disease (NAFLD)].

As described in the Examples below, genome wide analysis (RNA sequencing) and untargeted lipid analysis using LC-MS-MS revealed substantial increases in cholesterol and lipid abundance in the spinal cord of PAR1 and PAR2 knockout mice. Since cholesterol and lipids are significant components of myelin and neural membranes, including synapses, these data collectively support a new biological model in which PAR1 and/or PAR2 activation serves as a negative regulator of cholesterol and lipid. production in the CNS and, most likely, in all organs and tissues of the body. in this model, blocking the activity of PAR1 or PAR2 increases cholesterol and lipid synthesis. Therefore, PAR1 and PAR2 represent new targets for modulating cholesterol and lipid production, with receptor activation serving to promote reductions in lipid and cholesterol levels, and receptor inhibiting serving to increase levels of lipids and cholesterol.

This document therefore provides methods that can include administering, to a mammal identified as having a condition characterized by impaired lipid production, impaired cholesterol production, or both, an inhibitor of PAR1 and/or PAR2. In some cases, the methods can further include identifying a mammal as having a condition characterized by impaired lipid production, impaired cholesterol production, or both. Any suitable mammal can be treated using the methods provided herein. The mammal can be, for example, a human, a non-human primate, a cow, a horse, a pig, a sheep, a goat, a cat, to a dog, a mouse, or a rat. In some cases, the mammal to be treated with a PAR1 and/or PAR2 inhibitor can be identified as having a condition such as Alexander disease, metachromatic leukodystrophy, Krabbe disease, adrenoleukodystrophy, Canavan disease, Pelizaeus-Merzbacher disease, cerebrotenineous xanthomatosis, hypomyelinating leukodystrophy type 7, Refsum disease, or a congenital lipidosis.

Any suitable PAR1 and/or PAR2 inhibitor can be used. In some cases, for example, a small molecule such as Vorapaxar (also known as SCH530348) can be administered to block PAR1. Other non-limiting examples of small molecule PAR1 inhibitors include SCH79797, parmodulins (e.g., ML161 and NRD21), and atopaxar (E5555). Examples of small molecule PAR2 inhibitors include, without limitation, GB88, I-191, AZ3451, AZ2623, AZ0107, and AZ8838. Additional examples of PAR1 and/or PAR2 blocking molecules include function-blocking antibodies or antibody fragments (e.g., Fab′ fragments, F(ab′)₂ fragments, or scFv fragments), antisense molecules, interfering RNA [RNAi, including short interfering RNA (siRNA) and short hairpin RNA (shRNA)], and pepducins, any of which can be used to increase lipid and cholesterol production as described herein. Chimeric antibodies and humanized antibodies made from non-human (e.g., mouse, rat, gerbil, or hamster) antibodies also can be useful. Chimeric and humanized monoclonal antibodies can be produced by recombinant DNA techniques known in the art, for example, using methods described in U.S. Pat. Nos. 4,816,567; 5,482,856; 5,565,332; 6,054,297; and 6,808,901.

Antisense oligonucleotides typically are at least 8 nucleotides in length (e.g., about 8, 9, 10, 11, 12, 13, 14, 15, 16, 17, 18, 19, 20, 10 to 10, 15 to 20, 18 to 25, or 20 to 50 nucleotides in length) and can hybridize to a PAR1 or PAR2 transcript. In some cases, an antisense molecule greater than 50 nucleotides in length can be used, including a full-length PAR1 or PAR2 mRNA. An “oligonucleotide” is an oligomer or polymer of ribonucleic acid (RNA) or deoxyribonucleic acid (DNA) or analogs thereof. Nucleic acid analogs can be modified at the base moiety, sugar moiety, or phosphate backbone to improve, for example, stability, hybridization, or solubility of a nucleic acid. Methods for synthesizing antisense oligonucleotides include, for example, solid phase synthesis techniques. Equipment for such synthesis is commercially available from several vendors including, for example, Applied Biosystems (Foster City, Calif.). Alternatively, expression vectors that contain a regulatory element that directs production of an antisense transcript can be used to produce antisense molecules.

Antisense oligonucleotides can bind to a nucleic acid encoding PAR1, including DNA encoding PAR1 RNA (including pre-mRNA and mRNA) transcribed from such DNA, and also cDNA derived from such RNA, under physiological conditions (i.e., physiological pH and ionic strength). The sequence of an antisense oligonucleotide need not be 100% complementary to that of its target nucleic acid in order to be hybridizable under physiological conditions. Antisense oligonucleotides can hybridize under physiological conditions when binding of the oligonucleotide to the PAR1 or PAR2 nucleic acid interferes with the normal function of the PAR1 or PAR2 nucleic acid, and non-specific binding to non-target sequences is minimal.

Target sites for PAR1 or PAR2 antisense oligonucleotides can include the regions encompassing the translation initiation or termination codon of the open reading frame (ORF) of the gene. In addition, the ORF can be targeted effectively in antisense technology, as can the 5′ and 3′ untranslated regions. In some cases, antisense oligonucleotides can be directed at intron regions or intron-exon junction regions. Further criteria that can he applied to the design of antisense oligonucleotides include, for example, the lack of predicted secondary structure of a potential antisense oligonucleotide, an appropriate G and C nucleotide content (e.g., about 50%), and the absence of sequence motifs such as single nucleotide repeats (e.g., GGGG runs). The effectiveness of antisense oligonucleotides at modulating expression of a PAR1 or PAR2 nucleic acid can he evaluated by measuring levels of the PAR1 mRNA or polypeptide (e.g., by Northern blotting, RT-PCR, Western blotting, ELISA, or immunohistochemical staining).

A representative human PAR1 mRNA sequence is set forth in SEQ ID NO:1, with the coding sequence underlined:

(SEQ ID NO: 1)
aaccgccccagacacagcgctcgccgagggtcgcttggaccctgatcttacccgtgggcaccctgcgctctgcctgccgcgaa
gaccggctccccgacccgcagaagtcaggagagagggtgaagcggagcagcccgaggcggggcagcctcccggagcag
cgccgcgcagagcccgggacaatggggccgccgccgctgctgctggtggccgcctgcttcagtctgtgcgccccgctgttgt
ctgcccgcacccgggcccgcaggccagaatcaaaagcaacaaatgccaccttagatccccggtcatttcttctcaggaacccc
aatgataaatatgaaccattttgggaggatgaggagaaaaatgaaagtcggttaactgaatacagattagtctccatcaataaaag
cagtcctcttcaaaaacaacttcctgcattcatctcagaagatgcctccggatatttgaccagctcctggctgacactctttgtcccat
ctgtgtacaccggagtgtttgtagtcagcctcccactaaacatcatggccatcgttgtcttcatcctgaaaatgaaggtcaagaagc
cggcggtggtgtacatgctgcacctggccacggcagatgtcctgtttgtgtctgtgctcccctttaagatcagctattacttttccgg
cagtgattggcagtttgggtctgaattgtgtcgcttcgtcactgcagcattttactgtaacatgtacgcctctatcttgctcatgacagt
cataagcattgacccgtttctcgctgtggtgtatcccatgcagtccctctcctggcgtactctgggaaggccttccttcacttgtctg
gccatctgggctttggccatcgcaggggtagtgcctctgctcctcaaggagcaaaccatccaggtgcccgggctcaacatcact
acctctcatgatstcctcaatgaaaccctgctcgaaggctactatccctactacttctcagccttctctgctgtcttcttttttctcccgc
tgatcatttccacggtctgttatgtgtctatcattcgatgtcttagctcttccgcagttgccaaccgcagcaagaagtcccgggctttg
ttcctgtcagctgctgttttctgcatcttcatcatttgcttcggacccacaaacgtcctcctgattgcgcattactcattcctttctcacac
ttccaccacagaggctccctactttgcctacctcctctgtgtctgtgtcagcagcataagctgctgcatcgaccccctaatttactatt
acgcttcctctgagtgccagaggtacgtctacagtatcttatgctgcaaagaaagttccgatcccagcagttataacagcagtggg
cagttgatggcaagtaaaatggatacctgctctagtaacctgaataacagcatatacaaaaagctgttaacttaggaaaagggact
gctgggaggttaaaaagaaaagtttataaaagtgaataacctgaggattctattagtccccacccaaactttattgattcacctccta
aaacaacagatgtacgacttgcatacctgctttttatgggagctgtcaagcatgtatttttgtcaattaccagaaagataacaggacg
agatgacggtgttattccaagggaatattgccaatgctacagtaataaatgaatgtcacttctggatatagctaggtgacatatacat
acttacatgtgtgtatatgtagatgtatgcacacacatatattatttgcagtgcagtatagaataggcactttaaaacactctttccccg
caccccagcaattatgaaaataatctctgattccctgatttaatatgcaaagtctaggttggtagagtttagccctgaacatttcatgg
tgttcatcaacagtgagagactccatagtttgggcttgtaccacttttgcaaataagtgtattttgaaattgtttgacggcaaggtttaa
gttattaagaggtaagacttagtactatctgtgcgtagaagttctagtgttttcaattttaaacatatccaagtttgaattcctaaaattat
ggaaacagatgaaaagcctctgttttgatatgggtagtattttttacattttacacactgtacacataagccaaaactgagcataagtc
ctctagtgaatgtaggctggctttcagagtaggctattcctgagagctgcatgtgtccgcccccgatggaggactccaggcagca
gacacatgccagggccatgtcagacacagattggccagaaaccttcctgctgagcctcacagcagtgagactggggccactac
atttgctccatcctcctgggattggctgtgaactgatcatgtttatgagaaactggcaaagcagaatgtgatatcctaggaggtaat
gaccatgaaagacttctctacccatcttaaaaacaacgaaagaaggcatggacttctggatgcccatccactgggtgtaaacaca
tctagtagttgttctgaaatgtcagttctgatatggaagcacccattatgcgctgtggccactccaataggtgctgagtgtacagagt
ggaataagacagagacctgccctcaagagcaaagtagatcatgcatagagtgtgatgtatgtgtaataaatatgtttcacacaaac
aaggcctgtcagctaaagaagtttgaacatttgggttactatttcttgtggttataacttaatgaaaacaatgcagtacaggacatata
ttttttaaaataagtctgatttaattgggcactatttatttacaaatgttttgctcaatagattgctcaaatcaggttttcttttaagaatcaat
catgtcagtctgcttagaaataacagaagaaaatagaattgacattgaaatctaggaaaattattctataatttccatttacttaagact
taatgagaclttaaaagcaltttttaacctcctaagtatcaagtatagaaaatcttcatggaattcacaaagtaatttggaaattaggtt
gaaacatatctcttatcttacgaaaaaatggtagcattttaaacaaaatagaaagttgcaaggcaaatgtttatttaaaagagcaggc
caggcgcggtggctcacgcctgtaatcccagcactttgggaggctgaggcgggtggatcacgaggtcaggagatcgagacca
tcctggctaacacggtgaaacccgtctctactaaaaatgcaaaaaaaattagccgggcgtggtggcaggcacctgtagtcccag
ctactcgggaggctgaggcaggagactggcgtgaacccaggaggcggaccttgtagtgagccgagatcgcgccactgtgctc
cagcctgggcaacagagcaagactccatctcaaaaaataaaaataaataaaaaataaaaaaataaaagagcaaactatttccaa
ataccatagaataacttacataaaagtaatataactgtattgtaagtagaagctagcactggttttattaatttagtgactattcattttat
ctaaatcagtgaagatttactgtcattgtttattagtctgtatatattaaaatatgatatcattaatgtacttacaaaatagtatgtcactgtt
tttatgttcattcttaaaaacataacctgtattaataaatgtgaacatttgcttggta

A representative human PAR2 mRNA sequence is set forth in SEQ ID NO:2, with the coding sequence underlined:

(SEQ ID NO: 2)
tcggtgcgtccagtggagctctgagtttcgaatcggtggcggcggattccccgcgcgcccggcgtcggggcttccaggaggat
gcggagccccagcgcggcgtggctgctcggggccgccatcctgctagcagcctctctctcctgcagtggcaccatccaagga
accaatagatcctctaaaggaagaagccttattggtaaggttgatggcacatcccacgtcactggaaaaggagttacagttgaaa
cagtcttttctgtggatgagttttctgcatctgtcctcactggaaaactgaccactgtcttccttccaattgtctacacaattgtgtttgtg
gtgggtttgccaagtaacggcatggccctgtgggtctttcttttccgaactaagaagaagcaccctgctgtgatttacatggccaat
ctggccttggctgacctcctctctgtcatctggttccccttgaagattgcctatcacatacatggcaacaactcgatttatggggaag
ctctttgtaatgtgcttattggctttttctatggcaacatgtactgttccattctcttcatgacctgcctcagtgtgcagaggtattgggtc
atcotgaaccccatggggcactccaggaagaaggcaaacattgccattggcatctccctcgcaatatggctgctgattctcctgg
tcaccatccctttgtatgtcgtgaagcagaccatcttcattcctgccctgaacatcacgacctgtcatgatgttttccctgagcagctc
ttggtgggagacatgttcaattacttcctctctctggccattggggtctttctgttcccagccttcctcacagcctctgcctatgtgctg
atgatcagaatgctccgatcttctgccatggatgaaaactcagagaagaaaaggaagagggccatcaaactcattgtcactgtcc
tggccatgtacctgatctgcttcactcctagtaaccttctgcttgtggtgcattattttctgattaagagccagggccagagccatgtc
tatgccctctacattgtagccctctgcctctctacccttaacagctgcatcgacccctttgtctattactttgtttcacatgatttcaggg
atcatgcaaagaacgctctcctttgccgaagtgtccgcactgtaaagcagatgcaagtatccctcacctcaaagaaacactccag
gaaatccagctcttactcttcaagttcaaccactgttaagacctcctattga

Single and double-stranded interfering RNA (RNAi, such as siRNA and shRNA) homologous to PAR1 or PAR2 DNA also can be used to reduce expression of PAR1 or PAR2 and consequently, activity of PAR1 or PAR2. See, e.g., U.S. Pat. No. 6,933,146; Fire et al., Nature 391:806-811, 1998; Romano and Masino, Mol. Microbial. 6:343-3353, 1992; Cogoni et al., EMBO J. 15:3153-3163, 1996; Cogoni and Masino, Nature 399:166-169, 1999; Misquitta and Paterson, Proc. Natl. Acad. Sci. USA 96:1451-1456, 1999; and Kennerdell and Carthew, Cell 95:1017-1026, 1998.

The sense and anti-sense RNA strands of RNAi can be individually constructed using chemical synthesis and enzymatic ligation reactions using procedures known in the art. For example, each strand can be chemically synthesized using naturally occurring nucleotides or nucleic acid analogs. The sense or anti-sense strand also can be produced biologically using an expression vector into which a target PAR1 or PAR2 sequence (full-length or a fragment) has been subcloned in a sense or anti-sense orientation. The sense and anti-sense RNA strands can he annealed in vitro before delivery of the dsRNA to cells. Alternatively, annealing can occur in vivo after the sense and anti-sense strands are sequentially delivered to the tumor vasculature or to tumor cells.

In some cases, a genetic approach can he used to knock down PAR1 or PAR2 gene function. For example, CRISPR/Cas-mediated genome editing, adeno-associated virus- (AAV-) mediated delivery of a knockdown vector (e.g., shRNAi), or other suitable means can be used. These approaches may be applied to a population of stem cells ex vivo, or can he used in a mammal per se. For example, in some cases, a population of stem cells that have been modified to have reduced PAR1 and/or PAR2 expression, as compared to corresponding wild type neural stem cells, can be used. For example, stem cells can be modified in vitro to contain a mutation in the PAR1 and/or PAR2 gene, such that PAR1 and/or PAR2 expression is reduced or even knocked out. Suitable types of stern cells include, without limitation, embryonic stem cells, induced pluripotent stein cells, bone marrow derived stem cells, mesenchymal stem cells, and neural stem cells. After delivery to a mammal, the stern cells can differentiate into neuronal cells and, due to their reduced level of PAR1 and/or PAR2 expression, can lead to increased lipid or cholesterol production.

An effective amount of a PAR1 and/or PAR2 inhibitor can be an amount sufficient to increase the level of one or more lipids and/or cholesterol in a mammal after to treatment by at least 5% (e.g., at least 10%, at least 20%, at least 25%, at least 50%, at least 75%, or at least 100%), as compared to the level of the one or more lipids or the level of cholesterol prior to treatment. Any appropriate method can be used to measure the level of one or more lipids or cholesterol in a mammal, or in a biological sample from a mammal to be treated as described herein. In some cases, for example, LC-MS can be used to determine the level of one or more lipids or the level of cholesterol in a sample from a mammal (e.g., a blood sample, a sample of cerebrospinal fluid, a spinal cord sample, or a solid tissue sample). In some cases, an effective amount of a PAR1 and/or PAR2 inhibitor can be an amount sufficient to reduce one or more symptoms of a disorder resulting from impaired production of lipids necessary for normal brain development (e.g., Alexander disease, metachromatic leukodystrophy, Krabbe disease, adrenoleukodystrophy, Canavan disease, Pelizaeus-Merzbacher disease, cerebrotenineous xanthomatosis, hypomyelinating leukodystrophy type 7, or Refsum disease).

In addition, this document provides methods that can include administering, to a mammal identified as having a condition characterized by excess lipid production, excess cholesterol production, or both, an activator of PAR1 and/or PAR2. In some cases, the method can further include identifying a mammal as having a condition characterized by excess lipid production, excess cholesterol production, or both. Again, any suitable mammal can be treated using the methods provided herein. The mammal can be, for example, a human, a non-human primate, a cow, a horse, a pig, a sheep, a goat, a cat, a dog, a mouse, or a rat. In some cases, the mammal to he treated with a PAR1 and/or PAR2 activator can be identified as having a condition such as, without limitation, Smith-Lemli-Opitz Syndrome, Gaudier, Niemann-Pick, Tay-Sachs, fatty liver disease, familial hypercholesterolemia, adrenoleukodystrophy, Fabry, Farber, Hunter, Hurler, Krabbe, Tangier, or NAFLD.

Any suitable PAR1 and/or PAR2 activator can be used. In some cases, for example, a peptide ligand that mimics a natural PAR1 or PAR2 ligand can be used. Examples of such peptide ligands include Thr-Phe-Leu-Leu-Arg (SEQ ID NO:3) for PAR1, and Ser-Leu-Ile-Gly-Lys-Val (SEQ ID NO:4) and 2-Furoyl-Leu-ile-Gly-Arg-Leu-Orn (SEQ ID NO:5) for PAR2. Other non-limiting examples of PAR1/PAR2 activators include enzymatic activators such as thrombin, plasmin, trypsin, kallikrein 6, and activating antibodies.

An effective amount of a PAR1 and/or PAR2 activator can be an amount sufficient to reduce the level of one or more lipids and/or cholesterol in a mammal after treatment by at least 5% (e.g., at least 10%, at least 20%, at least 25%, at least 50%, or at least 75%), as compared to the level of the one or more lipids and/or the level of cholesterol prior to treatment. In some cases, an effective amount of a PAR1 and/or PAR2 activator can be an amount that results in a reduction of one or more symptoms of a condition characterized by excess lipid production, excess cholesterol production, or both (e.g., Smith-Lemli-Opitz Syndrome, Gaucher, Niemann-Pick, Tay-Sachs, fatty liver disease, familial hypercholesterolemia, adrenoleukodystrophy, Fabry, Farber, Hunter, Hurler, Krabbe, Tangier, or NAFLD).

Any suitable route of treatment can be used. For example, a pharmaceutical composition containing an agent that inhibits or activates PAR1 and/or PAR2 in a mammal can be administered locally (e.g., to the brain or the CNS) or systemically. Administration can be, for example, oral, parenteral (e.g., by subcutaneous, intrathecal, intraventricular, intramuscular, or intraperitoneal injection, or by intravenous drip), or topical (e.g., transdermal, sublingual, ophthalmic, or intranasal), or can occur by a combination of such methods. Administration can be rapid (e.g., by injection) or can occur over a period of time (e.g., by slow infusion or administration of a slow release formulation).

In general, administration of an agent that inhibits or activates PAR1 and/or PAR2 in a mammal having a condition as described herein can increase or reduce the level of one or more lipids and/or cholesterol in the mammal, and can reduce at least one symptom of the condition. After identifying a mammal as having a need for treatment, the mammal can be treated with a composition containing a PAR1 and/or PAR2 inhibitor or activator. The composition can be administered to the mammal in any amount, at any frequency, and for any duration effective to achieve a desired outcome (e.g., to increase or decrease lipid and/or cholesterol levels) in the mammal. In some cases, for example, a composition can be administered to a mammal repeatedly (e.g., once or more than once a day, once or more than once a week, or once or more than once a month). The frequency of administration can remain constant or can be variable during the duration of treatment. Various factors can influence the frequency of administration. For example, the effective amount, duration of treatment, route of administration, and severity of the condition may require an increase or decrease in administration frequency.

In some cases, an effective amount of a composition containing a PAR1 and/or

PAR2 inhibitor or activator can be for example, from about 0.1 mg/kg to about 100 mg/kg (e.g., from about 0.01 mg/kg to about 0.05 mg/kg, from about 0.05 mg/kg to about 0.1 mg/kg, from about 0.1 mg/kg to about 0.5 mg.kg, from about 0.3 mg/kg to about 11 mg/kg, from about 1 mg/kg to about 10 mg/kg, from about 2 mg/kg to about 10 mg/kg, from about 5 mg/kg to about 10 mg/kg, from about 6 mg/kg to about 10 mg/kg, from about 6 mg/kg to about 8 mg/kg, or from about 7 mg/kg to about 9 mg/kg). In some cases, from about 100 μg to about 100 mg (e.g., from about 100 μg to about 1 mg, from about 1 mg to about 100 mg, from about 100 mg to about 250 mg, from about 250 mg to about 1000 mg, from about 300 mg to about 1000 mg, from about 400 mg to about 1000 mg, from about 100 mg to about 900 mg, from about 100 mg to about 800 mg, from about 400 mg to about 800 mg, or from about 500 mg to about 700 mg) of a PAR1 and/or PAR2 modulating agent (an activator or an inhibitor) can be administered to an average sized human (e.g., about 75-85 kg human) per administration (e.g., per daily or weekly administration) for about two to about twelve weeks or more.

If a mammal fails to respond to a particular amount, then the amount of the administered PAR1 and/or PAR2 inhibitor or activator can be increased by, for example, two fold. After receiving this higher amount, the mammal can be monitored for both responsiveness to the treatment and toxicity symptoms, and further adjustments can be made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration of a PAR1 and/or PAR2 activator or inhibitor can be any frequency that alters the production and/or levels of lipid and/or cholesterol in the mammal, without producing significant toxicity to the mammal. For example, the frequency of administration can be from about once a day to about once a month (e.g., from about once a week to about once every other week). The frequency of administration can remain constant or can be variable during the duration of treatment. A course of treatment with a composition containing a PAR1 and/or PAR2 activator or inhibitor can include rest periods. In some cases, a composition containing a PAR1 inhibitor (e.g., Vorapaxar) can be administered daily over a two-week period followed by a two-week rest period, and such a regimen can be repeated multiple times. As with the effective amount, various factors can influence the actual frequency of administration used for a particular application. In some cases, the effective amount, duration of treatment, use of multiple treatment agents, route of administration, and severity of the condition may require an increase or decrease in administration frequency.

An effective duration for administering a composition containing a PAR1 and/or PAR2 activator or inhibitor can be any duration that alters the levels and/or production of lipid and/or cholesterol in the mammal without producing significant toxicity to the mammal. In some cases, the effective duration can vary from several days to several months. In general, the effective duration can range from about six weeks to about six months or longer, even for years or for life. Multiple factors can influence the actual effective duration used for a particular treatment. For example, an effective duration can vary with the frequency of administration, effective amount, use of multiple treatment agents, route of administration, and severity of the condition being treated.

In some cases, a course of treatment and/or the severity of one or more symptoms related to the condition being treated can be monitored. Any appropriate method can be used to determine whether or not a mammal's lipid and/or cholesterol levels are altered. For example, a biological sample (e.g., a blood or tissue sample) can be assessed following administration of a PAR1 and/or PAR2 inhibitor or activator to determine if the treatment increased or reduced the level of one or more lipids or cholesterol in the sample, as compared to the level measured in a sample obtained from a control mammal not having the condition, or as compared to the level measured in a sample obtained from the mammal prior to treatment. Any appropriate method (e.g., LC-MS) can be used to measure the level of lipids and/or cholesterol.

The invention will be further described in the following examples, which do not limit the scope of the invention described in the claims.

### EXAMPLES

### Example 1—Regulatory Role of PAR1 in Cholesterol and Lipid Production

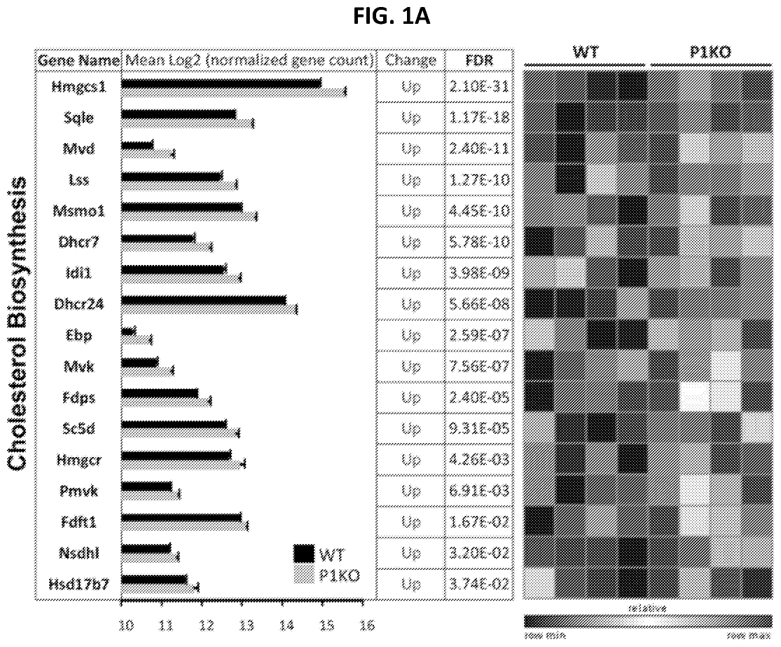

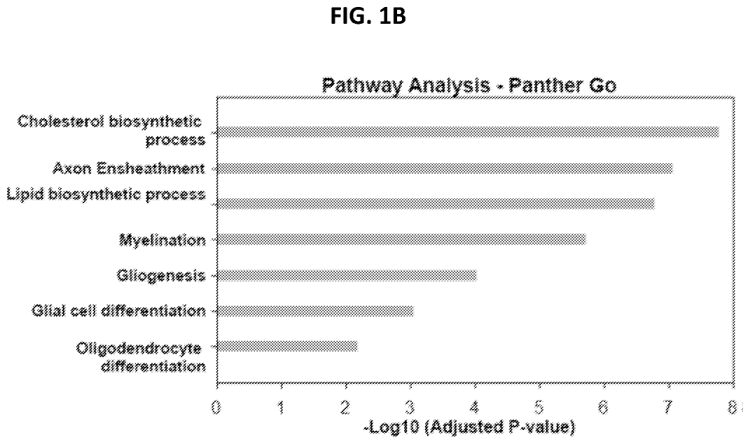

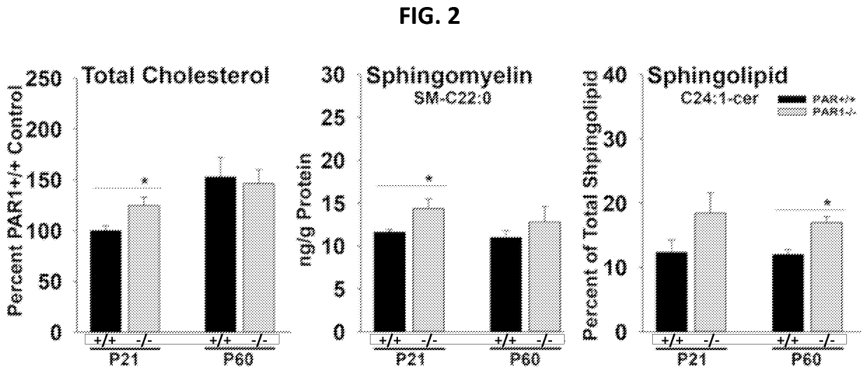

The spinal cord of PAR1 knockout mice contains higher levels of total cholesterol, and increases in expression of cholesterol and lipid synthesis intermediates (FIGS. 1A, 1B, and 2).

Studies are conducted to determine whether PAR1 is a regulator of cholesterol and lipids in the brain, the liver, and other organs, and to assess whether the increases in lipid and cholesterol production with PAR1 loss-of-function relate to increases in biosynthesis or instead reflect changes in degradation, efflux or influx. Additional studies implement a dynamic ¹³C-labeling gas chromatography-mass spectrometry (GC-MS) approach to determine whether PAR1 loss- or gain-of-function impacts cholesterol abundance by directly regulating biosynthesis in cultures of murine primary cortical neurons and the AML12 murine liver cell line.

For example, to determine whether PAR1 loss-of-function increases cholesterol and lipid levels across organ systems, cholesterol and lipid abundance are quantified in the brain and liver of wild type and PAR1 knockout mice using LC-MS and tissue samples collected from the same P60 mice used for the studies with results presented in (FIGS. 1A, 1B, and 2). Free and bound cholesterol, free fatty acids, sphingolipids, sphingomyelin, and galactocerebroside are specifically quantified. Quantitative PCR is used to determine whether expression of genes in the cholesterol and lipid synthesis pathways (e.g., DHCR7, Ugt8, and Fa2h) are elevated in brain and liver RNA as they are in the spinal cord. In addition, serum samples from the same wild type and PAR1 knockout mice are used to quantify HDL and LDL/VLDL using a quantitative fluorometric assay (Cholesterol Assay Kit ab65390; abeam, Cambridge, Mass.). Together, these results establish PAR1 as a fundamental regulator of cholesterol and lipid production across neural tissues and peripheral organ sites. The impact of PAR1 activation/inactivation is then evaluated in experimental models of lipid and cholesterol disorders (e.g., familial hypercholesterolemia, adrenoleukodystrophy, Fabry, Farber,

Hunter, Krabbe, Tangier, and NAFLD) and/or in conditions where increased lipids and cholesterol may enhance tissue regeneration (e.g., demyelinating lesions, neurotrauma, neurodegeneration, and congenital lipidoses).

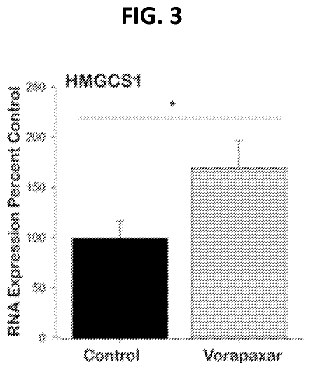

To assess the regulatory role of PAR1 gain- or loss-of-function in cholesterol biosynthesis, a GC-MS platform is utilized to quantify ¹³C-acetate incorporation into newly synthesized cholesterol in primary cultures of murine cortical neurons or in the murine hepatocyte cell line AML12. PAR1 loss-of-function is modeled using Vorapaxar, an FDA approved PAR1 small molecule inhibitor (FIG. 3) (Correa et al., J Thrombosis Thrombolysis 2019, 47(3):353-360; and Tsigkou et al., Curr Opin Pharmacol 2018, 39:43-52). PAR1 gain-of-function is modeled using a PAR1-activating peptide that mimics the tethered ligand of the receptor (Choi et al., Sci Rep 2018, 8(1):9360). The GC-MS assay for cholesterol biosynthesis quantification, established in the Mayo Metabolomics Core, includes standards for cholesterol and its synthesis intermediates (lansterol, zymosterol, desmosterol, and 7-dehydrocholesterol). The results demonstrated that PAR1 activity can regulate cholesterol biosynthesis, and suggesting strategies for therapeutic modulation.

Given the increases in cholesterol synthesis genes observed in the spinal cord of PAR1 knockout mice (FIGS. 1A and 1B), and in neurons treated with a PAR1 small molecule inhibitor (FIG. 3), PAR1 inhibition is likely to increase cholesterol biosynthesis while PAR1 activation promotes decreases. Alternatively, changes in cholesterol biosynthesis gene expression and overall abundance (FIGS. 1A, 1B, and 2) may occur by PAR1 effects on cholesterol efflux or uptake, and this is assessed as an alternative using cell-based assay kits. Additional studies apply similar dynamic labeling approaches to quantify the effect of PAR1 on lipid biosynthesis in vitro and on lipid and cholesterol biosynthesis in vivo.

### Example 2—PAR1 and PAR2 Regulation of Lipid and Cholesterol Biosynthesis

The studies described below demonstrated that genetic or pharmacologic inhibition of PAR1 function improved cholesterol and lipid synthesis in myelin and neural membranes across three models of central nervous system injury: (1) acute focal demyelination in the spinal cord by injection of lysolecithin into the ventral spinal cord (lysolecithin model), (2) chronic demyelination in the brain induced by feeding cupri zone chow (CPI model) to cause loss of myelin and oligodendrocytes in the corpus callosum, and (3) traumatic spinal cord injury (lateral compression spinal cord injury model). It is noted that parallel findings were observed for PAR2.

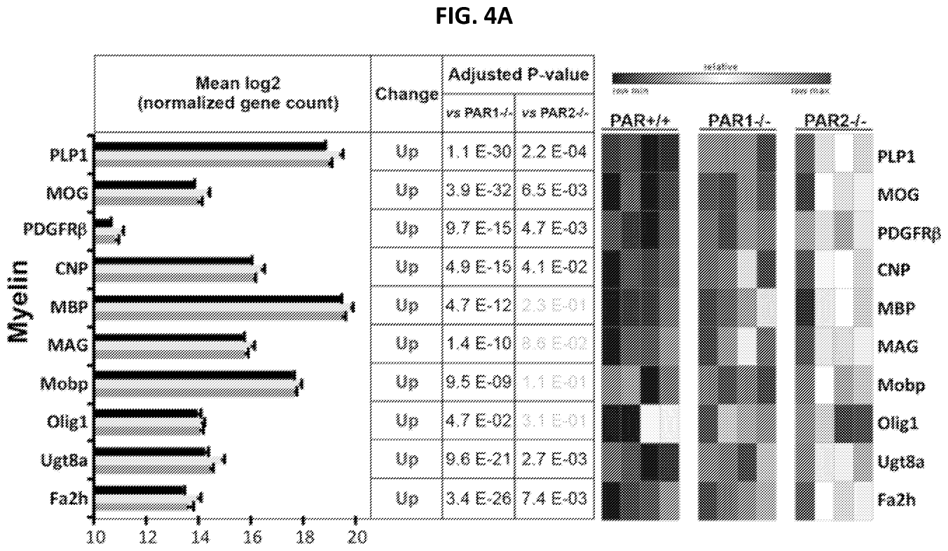

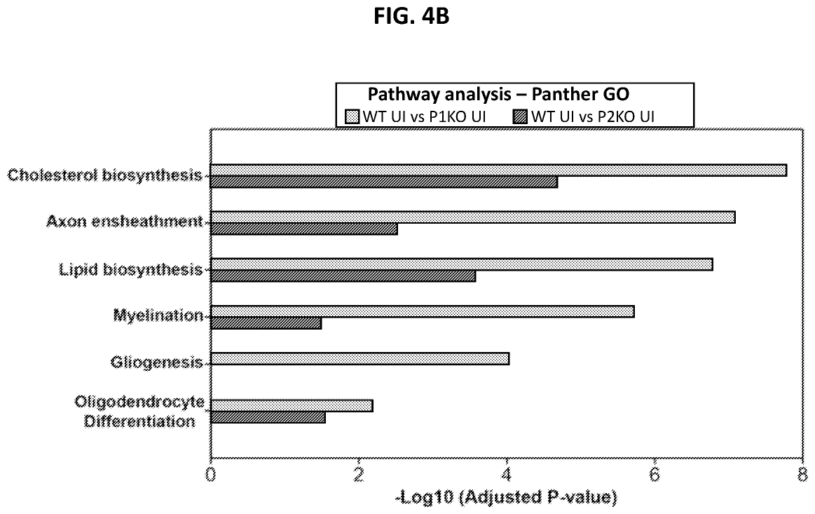

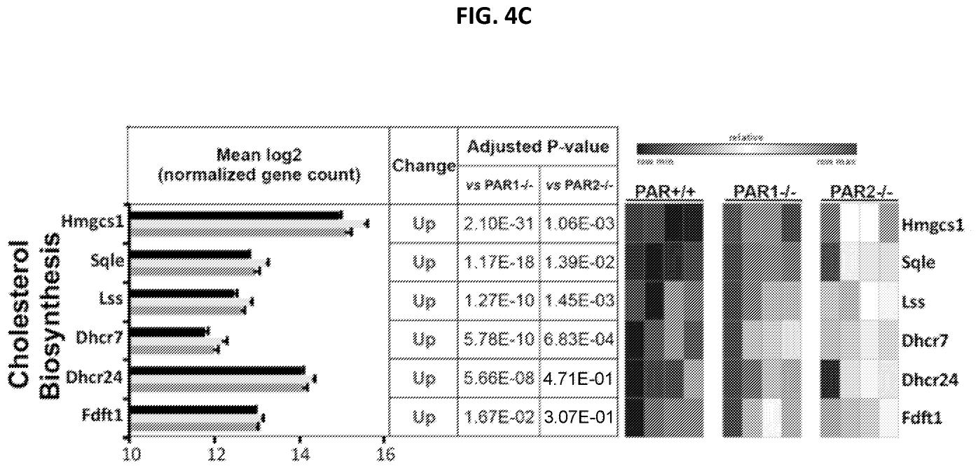

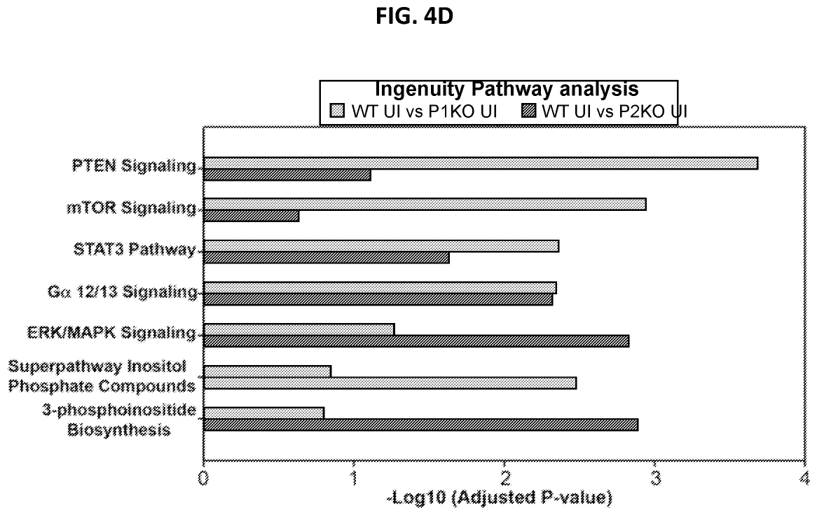

Differential gene expression analysis by whole genome RNA sequencing of the spinal cord of adult PAR1+/+, PAR1−/−, and or PAR2−/− mice demonstrated that PAR gene KO was associated with increased expression of genes critical for myelination, cholesterol and lipid biosynthesis. For example, major myelin proteins such as PLP1 and MBP were increased by PAR knockout (FIG. 4A). The Ugt8 (galactosyltransferase) and Fa2h (fatty acid 2-hydroxylase) genes also were upregulated (FIG. 4A); these are essential components of lipid synthesis pathways critical for membrane formation. Hmgcs1, which codes for an enzyme involved in production of HMG-CoA (a rate-limiting step in cholesterol biosynthesis) also was increased in PAR1−/− mice (FIG. 4C). DHCR7 encodes an enzyme involved in conversion of 7-dehydrocholesterol to cholesterol, and mutations in DHCR7 can disrupt cholesterol synthesis and manifest in Smith-Lemli-Opitz Syndrome. PANTHER GO Pathways highlighted key myelination events, including axon ensheathment, lipid biosynthesis, and oligodendrocyte differentiation (uninjured (UI) animals shown, FIG. 4B). Ingenuity Pathways analysis highlighted signaling intermediates in the ERK and AKT pathways (FIG. 4D).

Lipid quantification by LC-MS demonstrated that PAR1 and PAR2 knockout mice also exhibited higher levels of cholesterol and/or lipid synthesis in the spinal cord (FIG. 5). In particular, cholesterol and sphingomyelin were increased in the spinal cord of PAR−/− mice at P21, and sphingolipid was increased at P60. The spinal cord of PAR2−/−mice showed increases in sphingomyelin at P21 and P60 relative to PAR+/+ (*P<0.05, **P<0.01, ***P<0.001, NK).

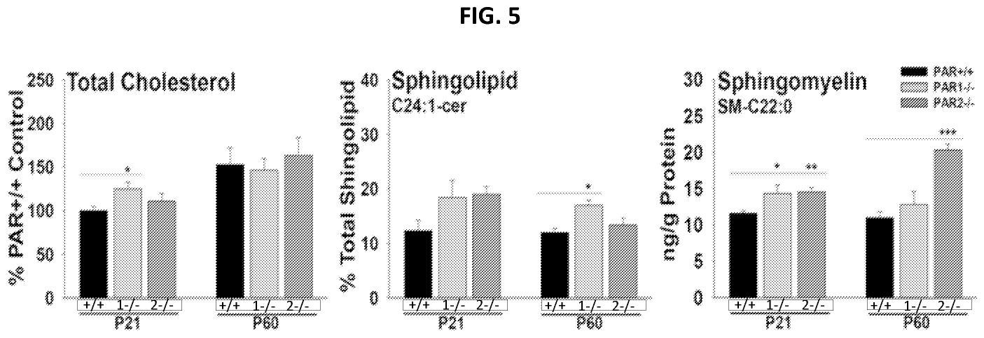

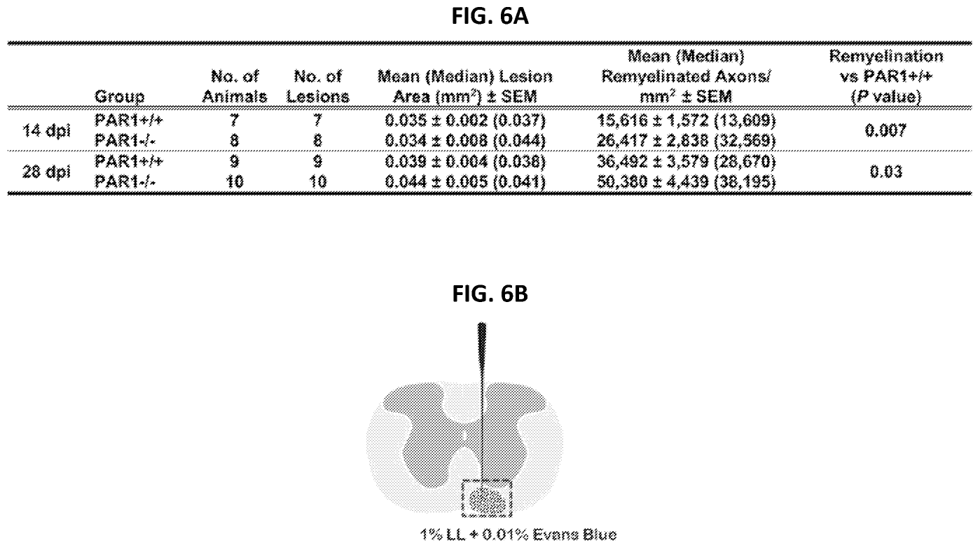

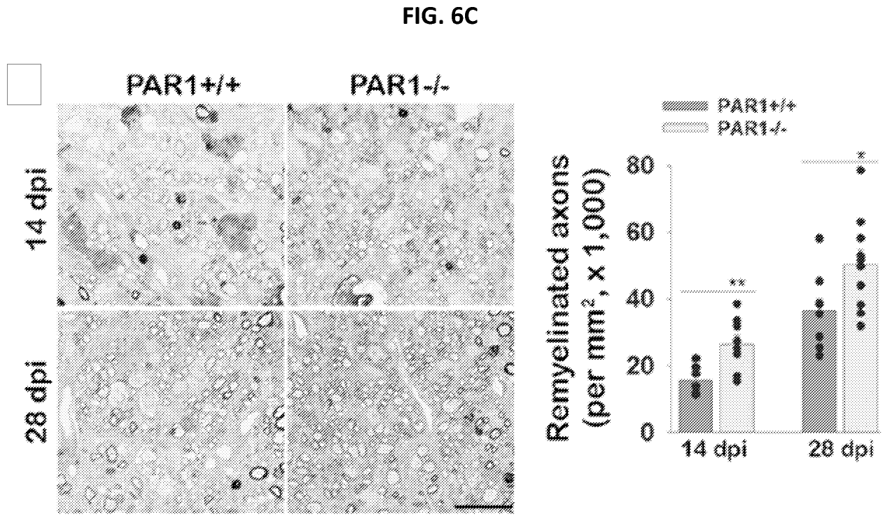

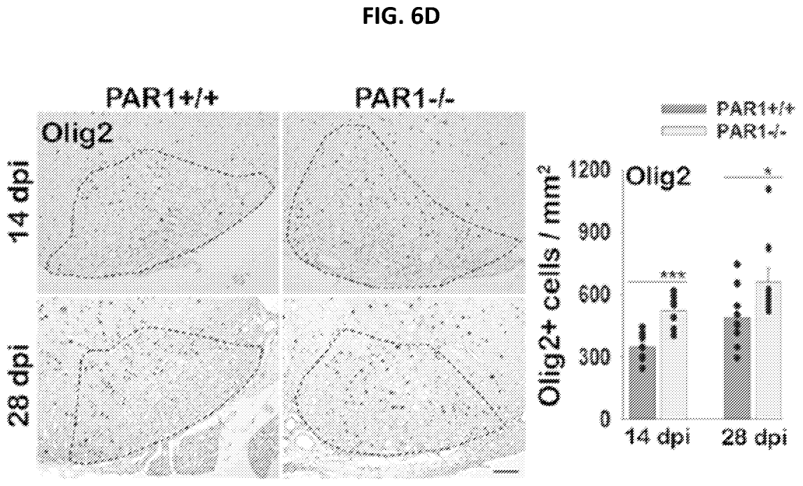

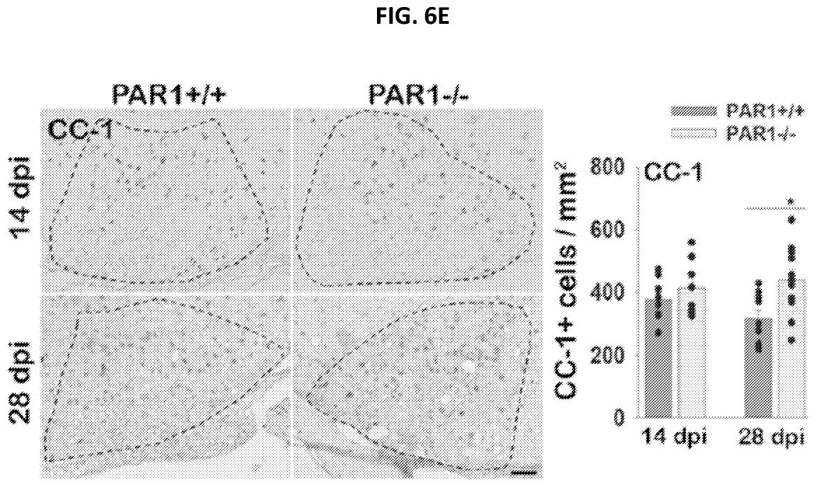

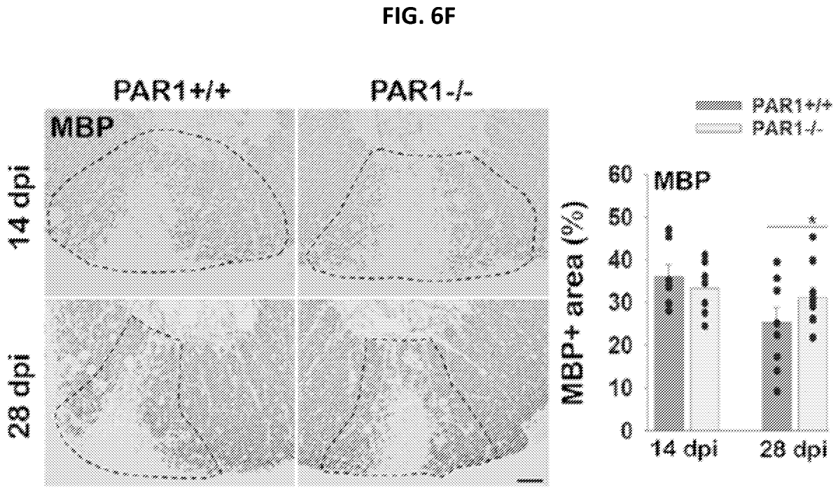

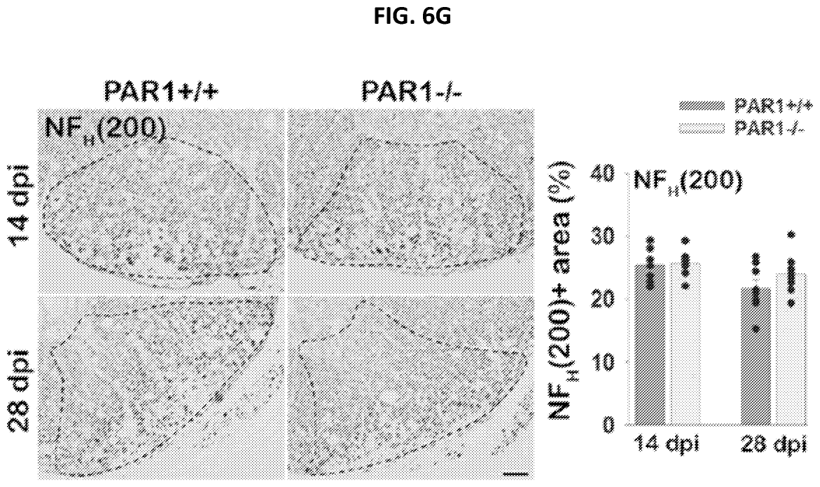

Studies using the lysolecithin model of focal demyelination showed that PAR1 knockout mice exhibited improved myelin regeneration (FIGS. 6A-6G). The mean numbers of remyelinated axons after focal lysolecithin-mediated demyelination of the ventral spinal cord white matter (FIG. 6B) in PAR+/+ and PAR1−/− mice are provided in FIG. 6A. The number of remyelinated axons was greater in PAR1 knockout mice at 14 or 28 days post injury (dpi) (P=0.007 and P=0.03, Student's t-test). The number of remyelinated axons was determined from representative paraphenylenediamine-stained thin sections (FIG. 6C). Moreover, PAR1 knockout mice exhibited improvements in the number of Olig2+ oligodendrocyte lineage cells at 14 and 28 dpi (FIG. 6D; P=0.001 and P=0.04) and increases in the number of CC-1+ mature oligodendrocytes at 28 dpi (FIG. 6E). By 28 dpi, higher levels of MBP were identified in the lesion area of PAR1 knockout mice (FIG. 6F; P=0.05). No significant changes were observed in neurofilaments across time or genotype (FIG. 6G).

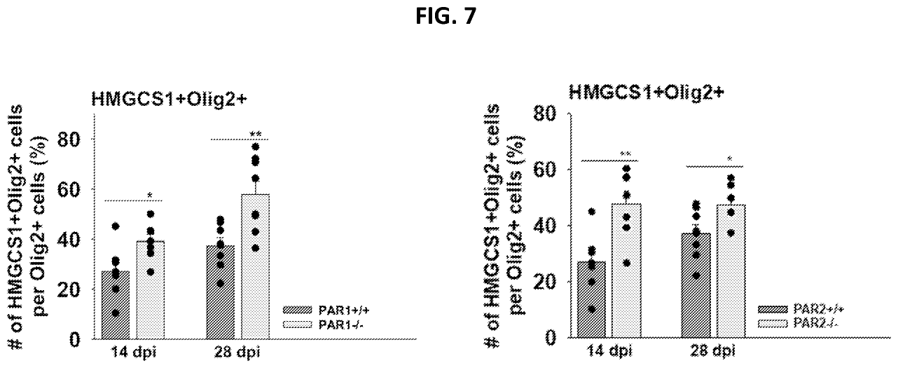

PAR1 knockout improved the cholesterol synthesis pathway in spinal cord oligodendrocytes after focal demyelinating injury (FIG. 7). In particular, Hmgcs1 (an essential regulatory enzyme in cholesterol production) was expressed at higher levels by Olig2+ oligodendrocytes in PAR1 knockout mice at 14 and 28 days post lysolecithin injury (dpi), as compared to wild type controls.

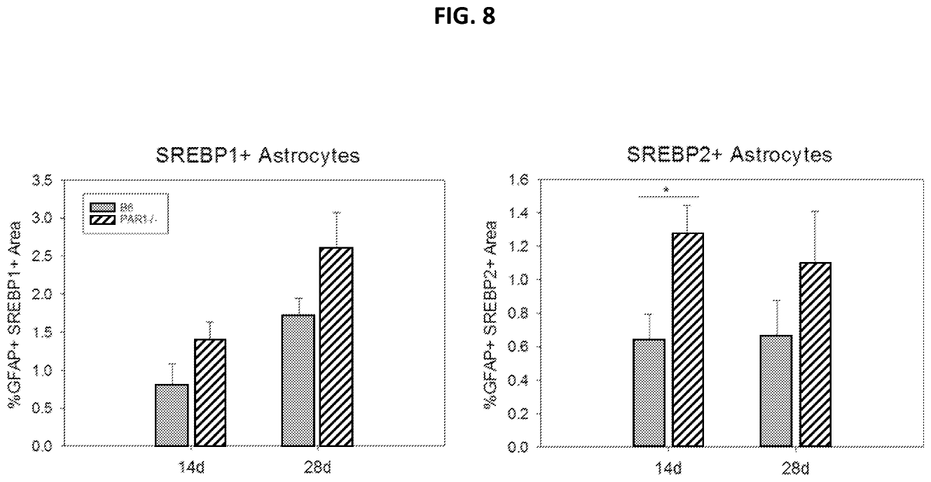

PAR1 knockout mice also showed increased expression of the master transcriptional regulators of cholesterol and lipid biosynthesis, SREBP1 (FIG. 8, left panel) and SREBP2 (FIG. 8, right panel) in astrocytes during myelin repair following lysolecithin-induced demyelination in the spinal cord. Spinal cords were stained for GFAP (an astrocyte marker), SREBP1, and SREBP2 at 14 or 28 days following lysolecithin-mediated focal demyelination. SREBP positivity was defined by measuring the percent of GFAP+ area colocalized with SREBP within the demyelinated lesion. SREBP2 expression in astrocytes was significantly increased in PAR1 knockout compared to wild-type controls at 14 days. Both SREBPs trended toward higher astrocytic expression at all time points in the context of PAR1 knockout (P<0.05).

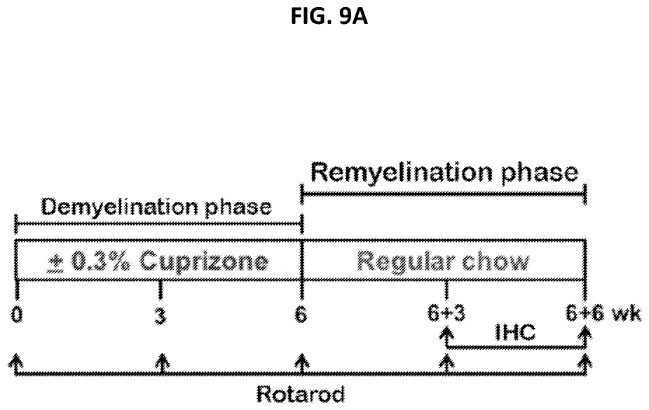

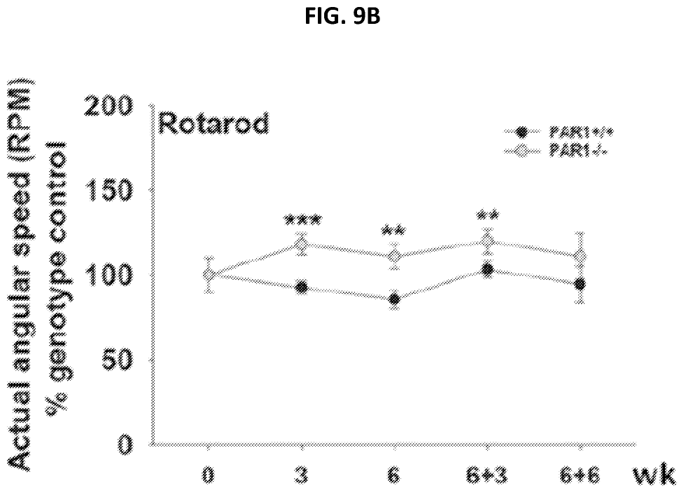

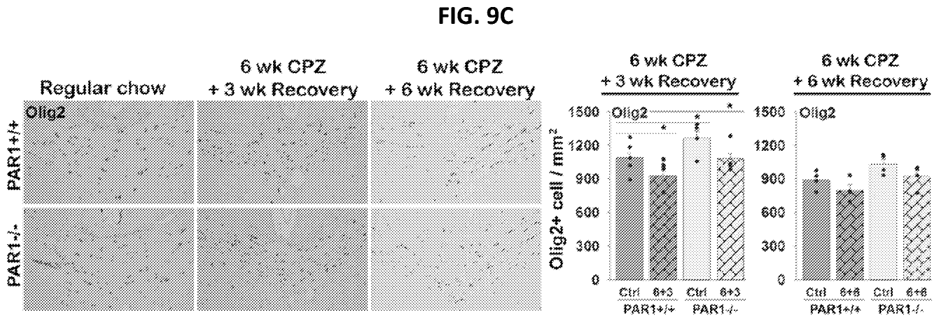

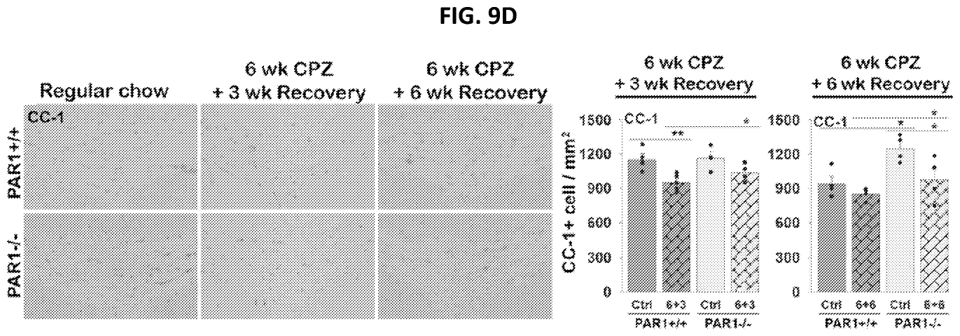

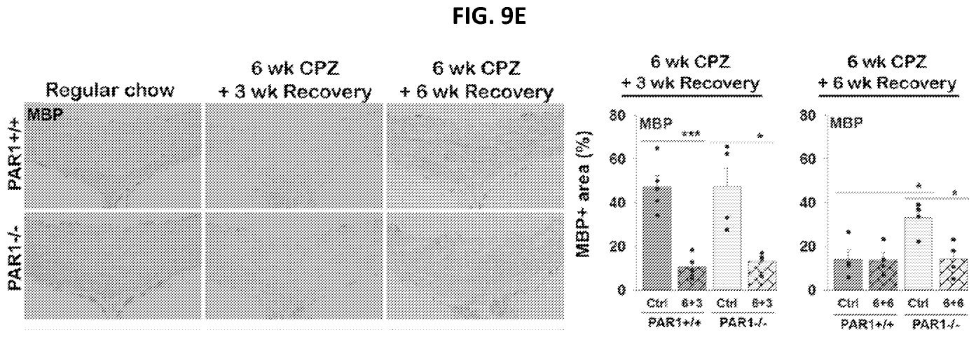

Further studies revealed that PAR1 knockout improved remyelination after CPZ withdrawal. The phases of demyelination and remyelination during and after CPZ feeding are depicted in FIG. 9A, with arrows indicating time points at which rotarod assessment (FIG. 9B) and immunohistochemistry (IHC) for markers of myelin injury and repair (FIGS. 9C-9F) were conducted. The impact of PAR1 knockout on remyelination was evaluated by feeding mice CPZ-laden chow for 6 weeks, followed by a period of “induced remyelination” upon CPZ withdrawal and feeding regular chow for an additional 3 (6+3) or 6 (6+6) week period. Motor function as assessed by angular speed at fall on an accelerating rotarod test, expressed as a percent of maximum at baseline for each genotype (FIG. 9B). These studies demonstrated less CPZ-related deficit at 3 (P=0.001) and 6 weeks (P=0.006) of CPZ-feeding and after an additional 3 weeks on normal chow (6+3) in PAR1 knockout mice (P=0.003). Representative images through the corpus callosum of mice after 3 or 6 weeks of remyelination (6+3 or 6+6) are shown in FIGS. 9C-9F. Quantification of markers of remyelination revealed that PAR1−/− mice exhibited greater increases in the number of oligodendrocyte lineage cells (Olig2+) (FIG. 9C) after 3 weeks on regular chow and in the number of mature oligodendrocytes (CC-1+) (FIG. 9B) at 3 and 6 weeks, compared to wild type (P≤0.05). The area stained for MBP (FIG. 9E) or neurofilament (NF) (FIG. 9F) after CPZ treatment was not altered by PAR1 knockout. However, at the 6+6 week time point, PAR1−/− mice consuming regular chow for the full period of the experiment showed higher counts of CC-1+ and higher levels of MBP and NF immunoreactivity in the corpus callosum (P≤0.04) relative to regular chow WT counterparts (FIGS. 9D, 9E, and 9F).

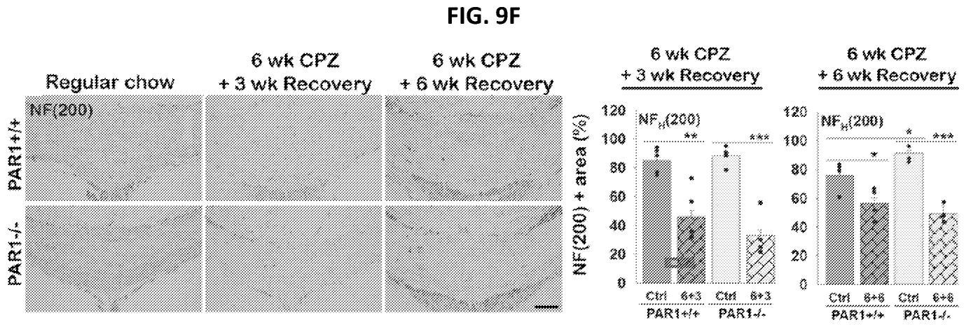

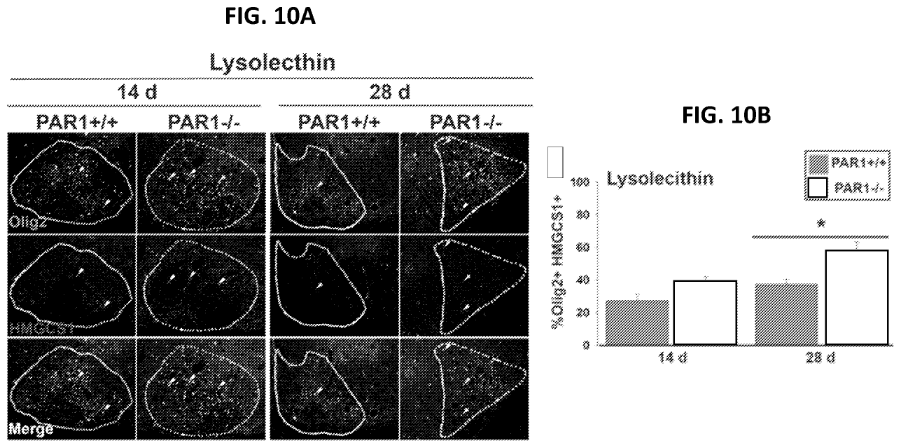

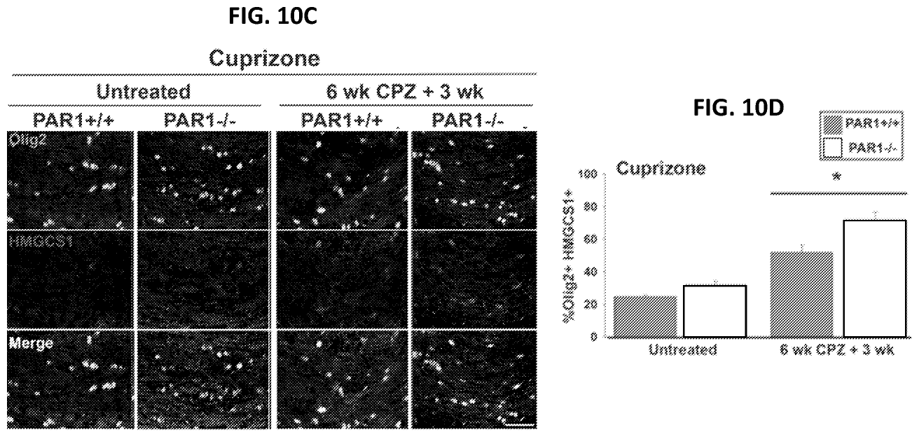

HMGCS1 is an essential regulatory enzyme in cholesterol production. Enhanced remyelination after cuprizone-mediated demyelination in PAR1 knockout mice was accompanied by increases in the percentage of HMGCS1+ oligodendrocytes after acute lysolecithin- or chronic cuprizone-mediated demyelination. Images taken of the ventrolateral spinal cord 14 or 28 days after lysolecithin injection revealed co-labelling of Olig2 for oligodendrocyte lineage cells and the cholesterol synthesis enzyme HMGCS1 (FIG. 10A), as well as areas of demyelination (outlined with the white dashed line in FIG. 10A). The PAR1−/− mice had significantly more HMGCS1-expressing oligodendrocytes at 28 days of remyelination (FIG. 10B). PAR1 knockout also improved the cholesterol synthesis pathway in CNS health and injury. For example, HMGCS1 was found to be expressed at higher levels by Olig2+ oligodendrocytes in PAR1 knockout mice prior to 6 weeks of CPZ mediated demyelination (Ctrl) and after 3 weeks on a regular diet to elicit myelin regeneration (6+3) (FIG. 10C). Results are plotted in FIG. 10D.

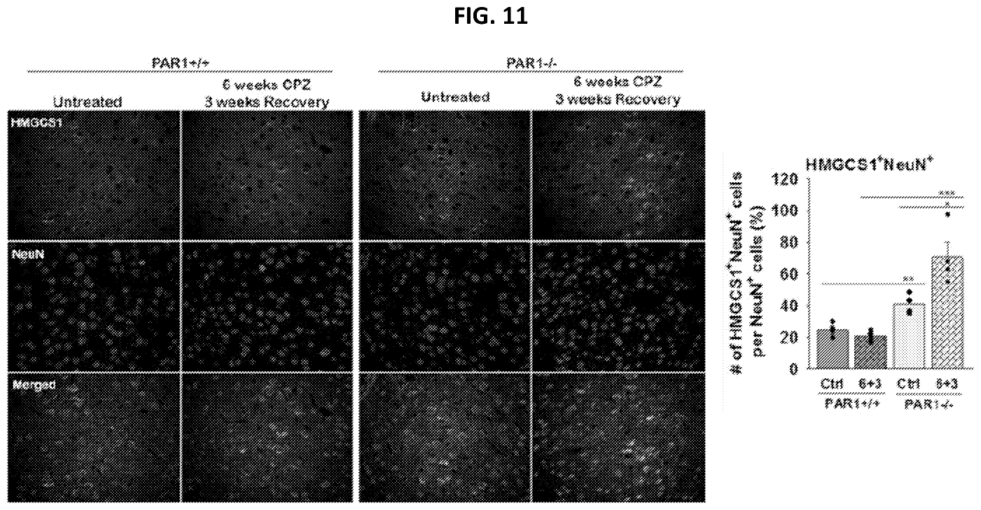

Hmgcs1 also was expressed at higher levels by NeuN+neurons in the cingulate cortex in PAR1 knockout mice prior to 6 weeks of CPZ mediated injury (Ctrl) and after 3 weeks on a regular diet to elicit repair (6+3) (FIG. 11). This suggested that blocking PAR1 increases cholesterol synthesis in neurons in the intact CNS and in response to CPZ mediated injury.

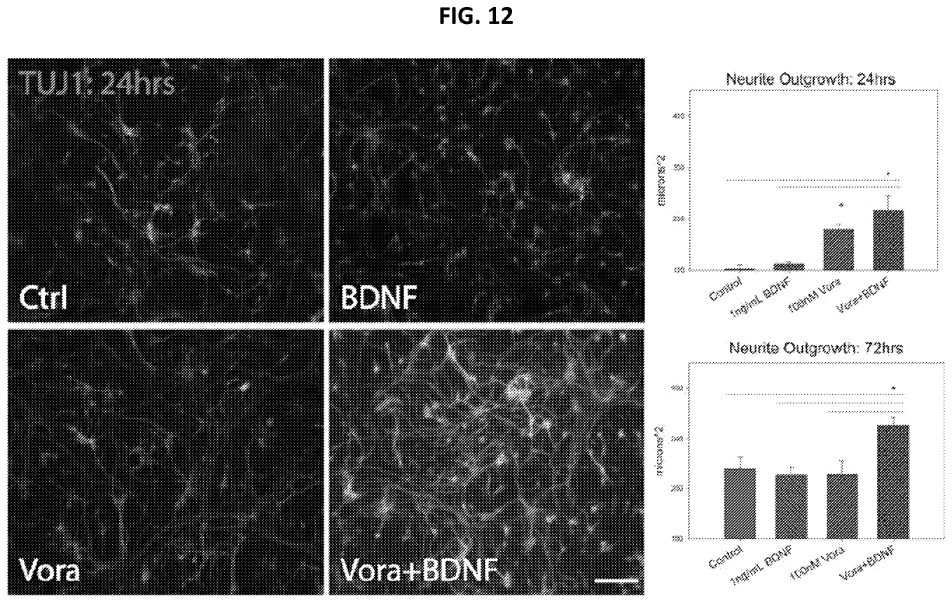

Inhibition of PAR1 signaling was found to accelerates neurite outgrowth in primary mouse cortical neurons (FIG. 12). Murine neurons were cultured for 24 hours before addition of the PAR1 inhibitor Vorapaxar, subtherapeutic growth factor BDNF, or both. Inhibition of PAR1 potentiated the effect of BDNF, significantly increasing production of neurites at both 24 and 72 hours of treatment. Neurite outgrowth was quantified by staining for TUJ1 (a cytoskeletal protein present in both axons and dendrites) and measuring TUJ1+ area (the average of 5 randomly selected fields per well, 6 wells per treatment). Production of lipids and cholesterol, major constituents of neuronal membranes, was required to grow both axons and dendrites.

Inhibition of PAR1 signaling also accelerated synaptogenesis in primary mouse cortical neurons (FIG. 13). Murine neurons were cultured in vitro for 24 hours before addition of the PAR1 inhibitor Vorapaxar, subtherapeutic growth factor BDNF, or both. Inhibition of PAR1 potentiated the effect of BDNF, significantly increasing production of synaptic densities at both 24 and 72 hours of treatment. Synapse density was quantified by staining for Homer, a scaffold protein concentrated at the post-synaptic densities by counting puncta in ImageJ. Synapse counts were normalized by total neurite area (TUJ1+ area) to determine the density of synapses on neurites (average of 5 randomly selected fields per well, 6 wells per treatment).

Inhibition of PAR1 signaling accelerated growth of new neurites following transection injury in vitro (FIG. 14). Murine neurons were cultured in vitro to confluence, and then a large wound was created by mechanically scratching the center of each field. The PAR1 inhibitor Vorapaxar, subtherapeutic growth factor BUNT, or both were added following injury. Inhibition of PAR1 potentiated the effect of BDNF, significantly increasing growth of new neurites through the injury zone, measured by TUJ1+ area in the scratch (the average of 3 randomly selected fields per well, 6 wells per treatment).

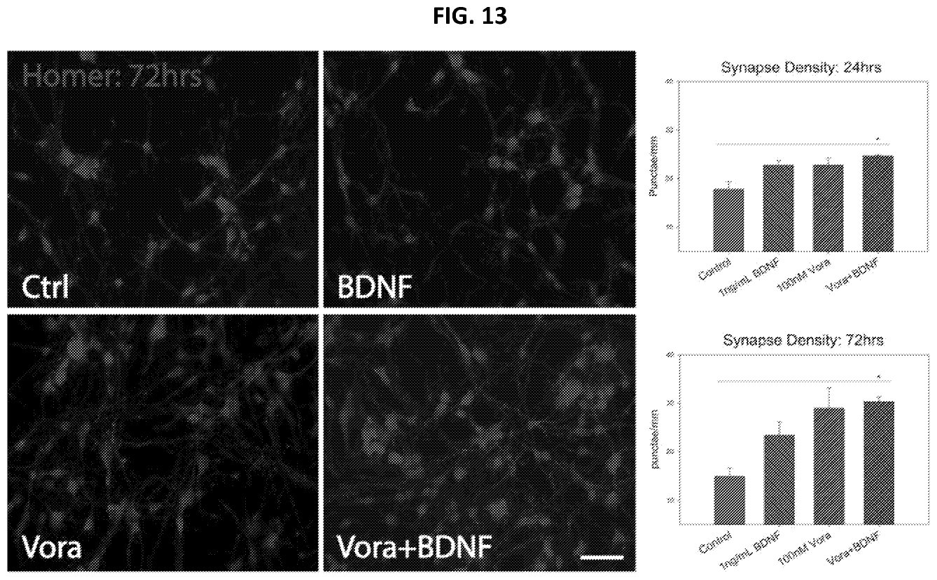

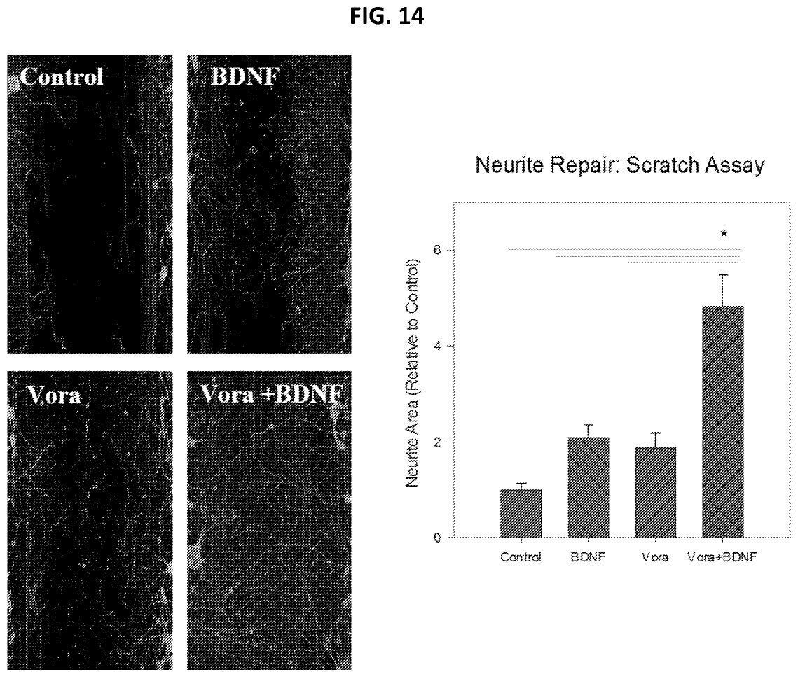

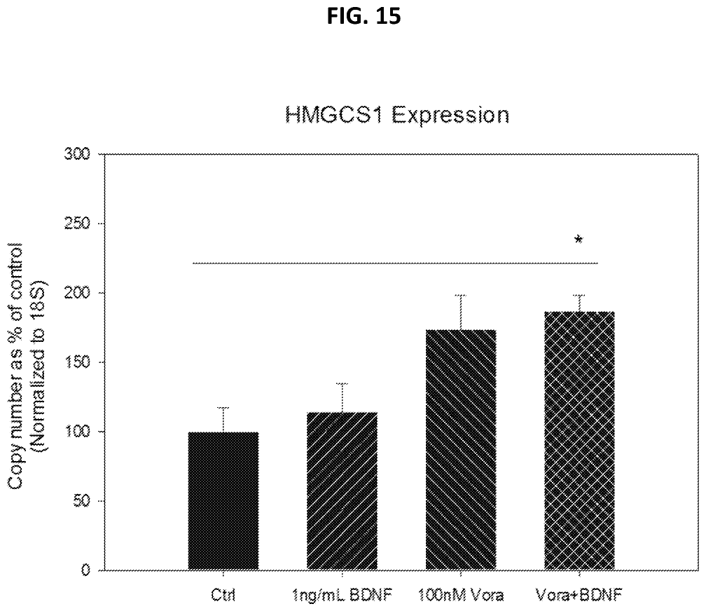

Inhibition of PAR1 by Vorapaxar was found to increase expression of the cholesterol synthesis enzyme HMGCS1 in primary murine cortical neurons (FIG. 15), as quantified by real-time quantitative PCR. HMGCS1 is responsible for production of HMGCoA for the rate-limiting step in cholesterol biosynthesis. Inhibition of PAR1 (with or without co-treatment with BDNF) increased the expression of HMGCS1 in cortical neurons.

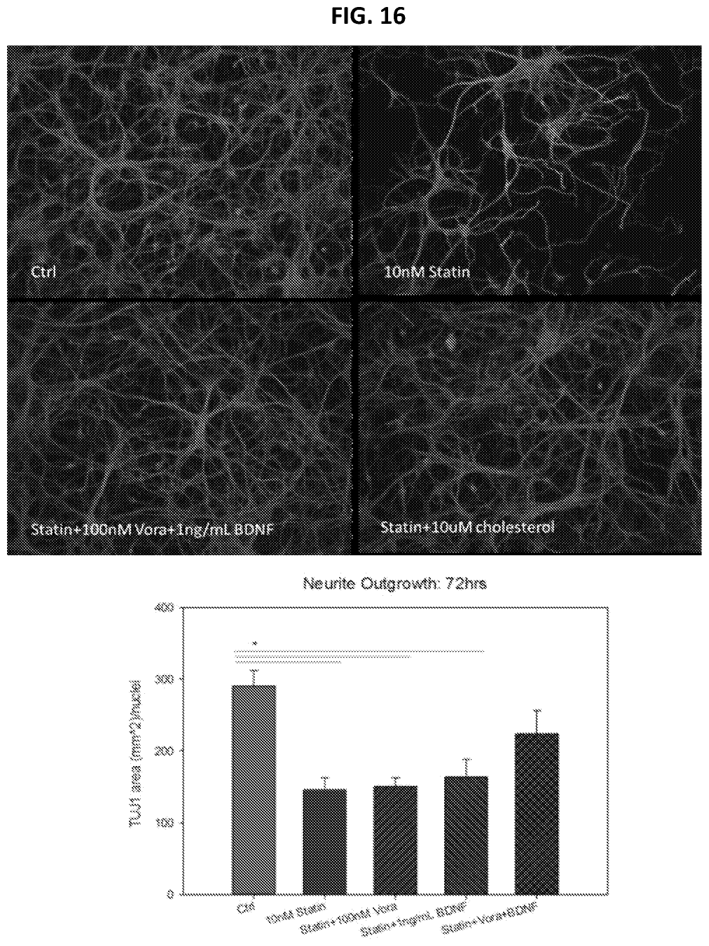

Inhibition of cholesterol production with statins (reversible inhibitors of HMG-CoA Reductase) resulted in diminished neurite outgrowth of primary mouse cortical neurons (FIG. 16), thus demonstrating the essential role of cholesterol production in neuron growth and development. Inhibition of PAR1 (with Vorapaxar) or growth factor (BDNF) alone did not result in recovery of neurite production and still produced a statistically significant decrease in neurite production compared to controls. However, co-treatment with both subtherapeutic BDNF with inhibition of PAR1 partially rescued. neurite production from statin treatment, and cotreated cultures were no longer statistically different from untreated controls. Neurite outgrowth was quantified by staining for TUJ1 (a cytoskeletal protein present in both axons and dendrites) and measuring TUJ1+ area (the average of 5 randomly selected fields per well, 6 wells per treatment).

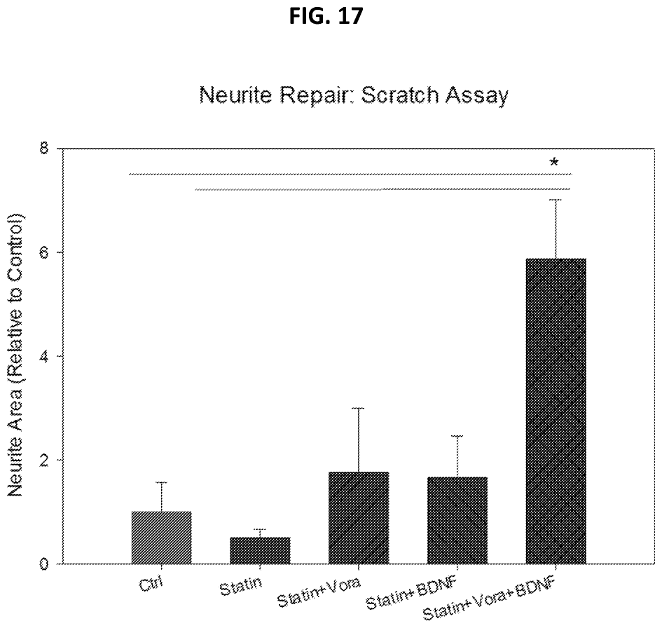

Inhibition of cholesterol production with statins also diminished neurite outgrowth of primary mouse cortical neurons following mechanical injury (FIG. 17), demonstrating the essential role of cholesterol production in neuron repair as well as development. Co-treatment with both subtherapeutic BDNF and inhibition of PAR1 rescued neurite repair from statin treatment and additionally increased repair even beyond untreated control cultures. Neurite outgrowth was quantified by staining for TUJ1 in the scratch zone and measuring TUJ1+ area (the average of 3 randomly selected fields per well, 6 wells per treatment).

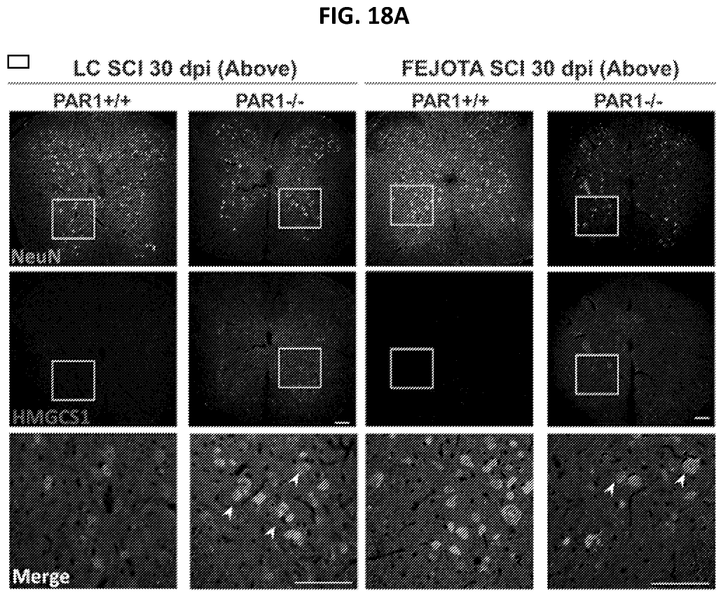

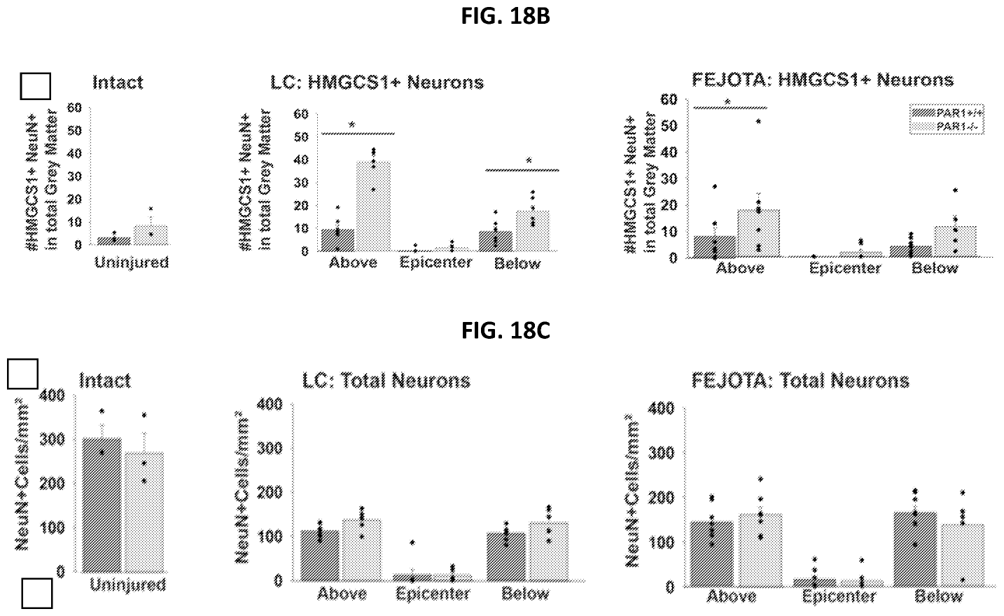

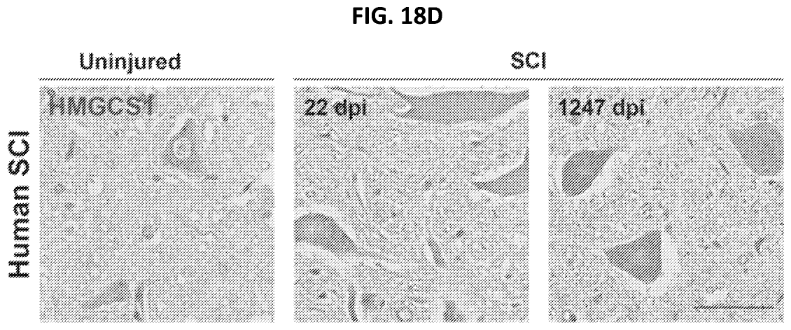

PAR1 knockout increased neuronal expression of cholesterol synthesis enzymes in mice following spinal cord injury by lateral compression or FEJOTA clip. After injury, cords were divided regionally by removing the epicenter of injury as well as tissue immediately above and below the injured region in wild type controls and PAR1 knockout mice. Slices from each region were stained for NeuN, a neuronal marker, and HMGCS1, a cholesterol synthesis enzyme. The number of HMGCS1+ neurons was counted for each slide. PAR1 knockout mice demonstrated a significantly increased number of neurons positive for HMGCS1 both above (FIGS. 18A and 18B) and below (FIG. 18B) the injured spinal cord, possibly indicative of enhanced ability for cholesterol production and therefore extension of new axons. In contrast, the total number of neurons in the spinal cord did not significantly differ by genotype with either injury (FIG. 18C). Expression of HMGCS1 by ventral horn motoneurons also was documented in the intact in human spinal cord and at subacute and chronic time points after traumatic SCI (FIG. 18D).

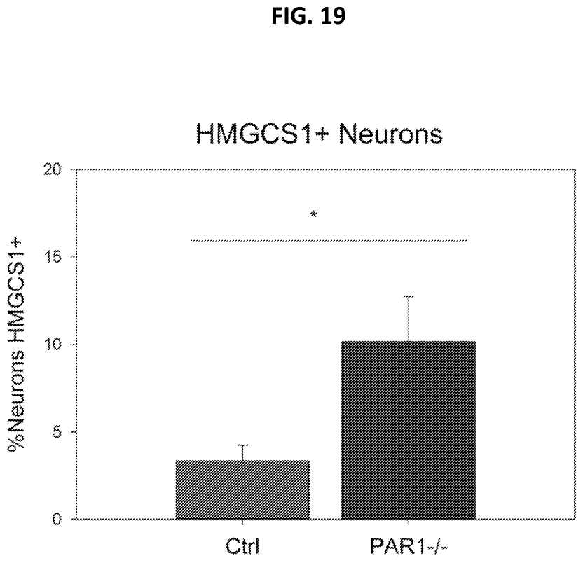

Studies also were conducted to demonstrate the percentage of neurons in PAR1−/− uninjured spinal cord and wild type controls that express HMGCS1. These studies demonstrated that a higher percentage of neurons in PAR1−/− uninjured spinal cord expressed HMGCS1, as compared to the wild type controls at baseline (FIG. 19). Spinal cord sections (5/animal) were imaged at levels corresponding to the SCI tissue (above, epicenter, below) and HMGCS1+ NeuN+ neurons were counted in ImageJ.

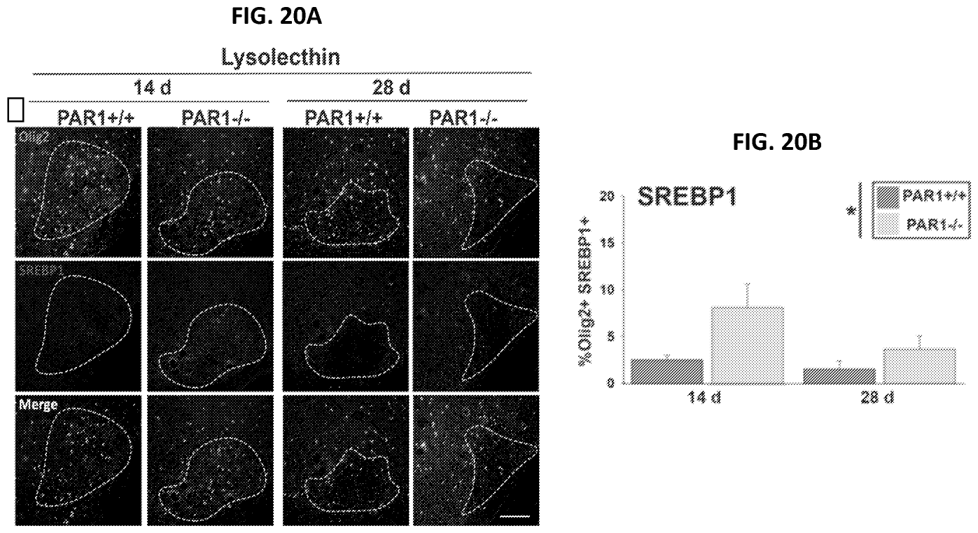

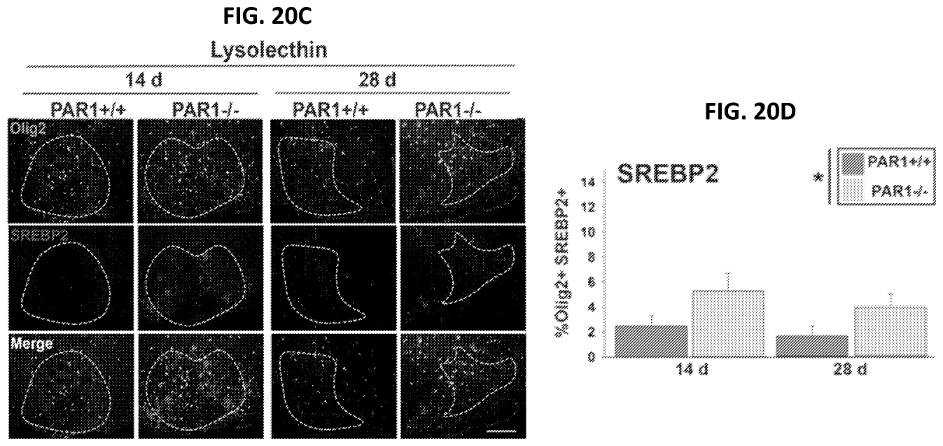

PAR1 inhibition also was found to increase oligodendrocyte expression of master regulators of lipid synthesis during remyelination in an acute model of myelin injury. In particular, enhanced remyelination after lysolecithin demyelination in PAR1 knockout mice was accompanied by increases in the number of SREBP1 oligodendrocyte lineage cells (FIGS. 20A and 20B; p=0.02). Two-Way ANOVA demonstrated a significant genotype difference, with PAR1−/− mice showing significantly more SREBP2+Olig2+ cells in remyelinating lesions (FIGS. 20C and 20D; p=0.04).

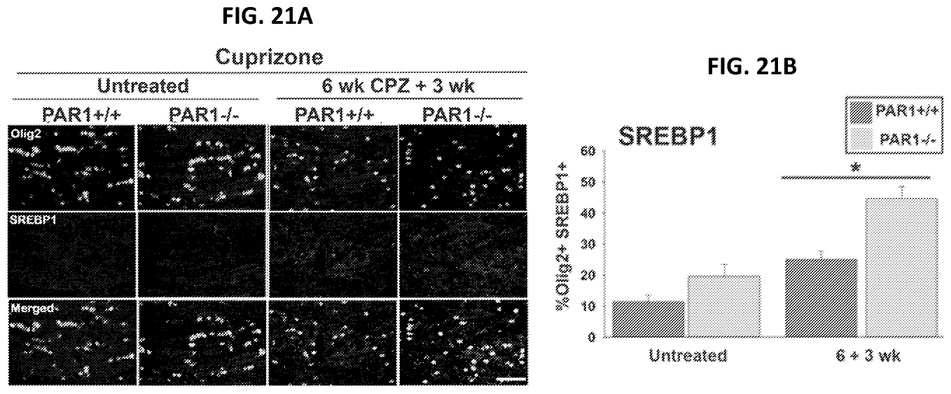

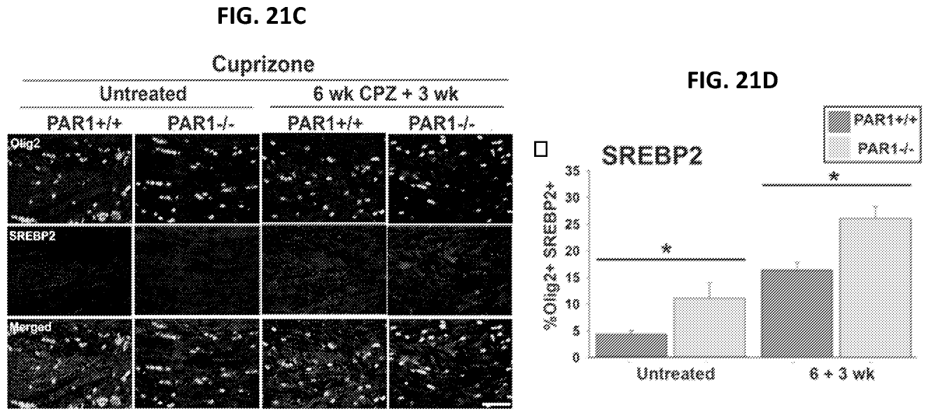

In addition, PAR1 inhibition increased oligodendrocyte expression of master regulators of lipid synthesis during remyelination in a chronic model of myelin injury. Enhanced remyelination after cuprizone-mediated demyelination in PAR1−/− mice was accompanied by increases in the number of SREBPI oligodendrocyte lineage cells in the corpus callosum of mice fed regular chow (untreated) or after 6 weeks of CPZ feeding followed by 3 weeks of regular chow consumption to induce myelin regeneration. Increases in SREBP1+ oligodendrocytes were observed during remyelination in both wild type and PAR1−/− mice (FIGS. 21A and 21B; p=0.006 and p<0.001, respectively), with the greatest increases observed in. PAR1−/− mice (p<0.001 compared to wild type). PAR1 knockout mice also showed a greater number of SREBP2+Olig2+ oligodendrocyte lineage cells than age matched controls not fed cuprizone (p=0.02) and during post-cuprizone remyelination (p=0.004) (FIGS. 21C and 21D)).

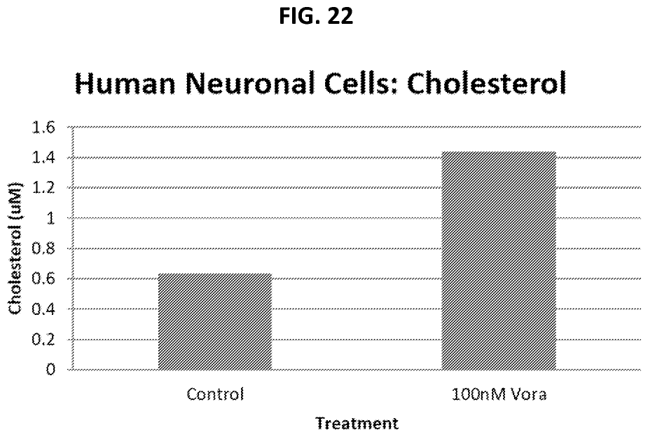

Finally, cholesterol levels were evaluated in a human neuronal cell line (SH-SY5Y cells) grown in the presence of the small molecule PAR1 inhibitor, Vorapaxar, for 72 hours. Vorapaxar-treated cells were found to contain more than twice the amount of cellular cholesterol measured in untreated cells (FIG. 22), as quantified by amplex red colorimetric assay.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
(a) identifying a mammal as having a condition characterized at least by impaired lipid production, impaired cholesterol production, or both impaired lipid production and impaired cholesterol production; and
(b) administering to said mammal an inhibitor of PAR1 and/or PAR2 in an amount effective to increase lipid production and/or cholesterol production in said mammal.

2. The method of claim 1, wherein said mammal is a human.

3. The method of claim 1, wherein said condition is selected from the group consisting of Alexander disease, metachromatic leukodystrophy, Krabbe disease, adrenoleukodystrophy, Canavan disease, Pelizaeus-Merzbacher disease, cerebrotenineous xanthomatosis, hypomyelinating leukodystrophy type 7, Refsum disease, and congenital lipidoses.

4. The method of claim 1, wherein said inhibitor of PAR1 and/or PAR2 is a small molecule.

5. The method of claim 4, wherein said small molecule is Vorapaxar, GB88, or a parmodulin.

6. A method comprising:
(a) identifying a mammal as having a condition characterized at least by excess lipid production, excess cholesterol production, or both excess lipid production and excess cholesterol production; and
(b) administering to said mammal an activator of PAR1 and/or PAR2 in an amount effective to reduce lipid production and/or cholesterol production in said mammal.

7. The method of claim 6, wherein said mammal is a human.

8. The method of claim 6, wherein said condition is selected from the group consisting of Smith-Lemli-Opitz Syndrome, Gaucher, Niemann-Pick, Tay-Sachs, fatty liver disease, familial hypercholesterolemia, adrenoleukodystrophy, Fabry, Farber, Hunter, Hurler, Krabbe, Tangier, acquired dyslipidemia, and non-alcoholic fatty liver disease (NAFLD).

9. The method of claim 6, wherein said activator of PAR1 and/or PAR2 is a peptide ligand.

10. The method of claim 9, wherein said peptide ligand is Thr-Phe-Leu-Leu-Arg (SEQ ID NO:3), Ser-Leu-Ile-Gly-Lys-Val (SEQ ID NO:4), or 2-Furoyl-Leu-Ile-Gly-Arg-Leu-Orn (SEQ ID NO:5).

11. A method comprising:
administering an inhibitor of PAR1 and/or PAR2 to a mammal identified as having a condition characterized at least by impaired lipid production, impaired cholesterol production, or both impaired lipid production and impaired cholesterol production,
wherein said inhibitor is administered in an amount effective to increase lipid and/or cholesterol production in said mammal.

12. The method of claim 11, wherein said mammal is a human.

13. The method of claim 11, wherein said condition is selected from the group consisting of Alexander disease, metachromatic leukodystrophy, Krabbe disease, adrenoleukodystrophy, Canavan disease, Pelizaeus-Merzbacher disease, cerebrotenineous xanthomatosis, hypomyelinating leukodystrophy type 7, Refsum disease, and congenital lipidoses.

14. The method of claim 11, wherein said inhibitor of PAR1 and/or PAR2 is a small molecule.

15. The method of claim 14, wherein said small molecule is Vorapaxar, GB88, or a parmoldulin.

16. A method comprising:
administering an activator of PAR1 and/or PAR2 to a mammal identified as having a condition characterized at least by excess lipid production, excess cholesterol production, or both excess lipid production and excess cholesterol production,
wherein said activator is administered in an amount effective to reduce lipid production and/or cholesterol production in said mammal.

17. The method of claim 16, wherein said mammal is a human.

18. The method of claim 16, wherein said condition is selected from the group consisting of Smith-Lemli-Opitz Syndrome, Gaucher, Niemann-Pick, Tay-Sachs, fatty liver disease, familial hypercholesterolemia, adrenoleukodystrophy, Fabry, Farber, Hunter, Hurler, Krabbe, Tangier, acquired dyslipidemia, and NAFLD.

19. The method of claim 16, wherein said activator of PAR1 and/or PAR2 is a peptide ligand.

20. The method of claim 19, wherein said peptide ligand is Thr-Phe-Leu-Leu-Arg (SEQ ID NO:3), Ser-Leu-Ile-Gly-Lys-Val (SEQ ID NO:4), or 2-Furoyl-Leu-Ile-Gly-Arg-Leu-Orn (SEQ ID NO:5).
